(19) United States
(12) Patent Application Publication
(10) Pub. No.: US 2008/0318942 A1
(43) Pub. Date: Dec. 25, 2008

(54) Fredericamycin Derivatives

(75) Inventors: Werner Simon, Huffelsheim (DE); Rajendra-Prasad Maskey, Heidelberg (DE); Hans-Falk Rasser, Hassloch (DE); Bernd Sontag, Sandhausen (DE); Peter Eckard, Schriesheim (DE); Iris- Grun-Wollny, Weinheim (DE); Friedrich Hansske, Weinheim (DE); Bjorn Friedrich Lindemann, Hamburg (DE)

Correspondence Address: CONNOLLY BOVE LODGE & HUTZ, LLP, P O BOX 2207, WILMINGTON, DE, 19899, US

(73) Assignees: BIOAGENCY AG, HAMBURG (DE); DISCOVERY PARTNERS INTERNATIONAL GMBH, HEIDELBERG (DE)

(21) Appl. No.: 12/065,108

(22) PCT Filed: Sep. 1, 2006
(86) PCT No.: PCT/DE2006/001534

§ 371 Date: Aug. 5, 2008

(30) Foreign Application Priority Data
Sep. 1, 2005 (DE) 10 2005 041 760.4
Feb. 9, 2006 (DE) 10 2006 005 936.0
Feb. 9, 2006 (DE) 10 2006 005 937.9

(51) Int. Cl. A61K 31/5377; C07D 221/20; A61K 31/438; A61P 33/00; A61P 17/00; A61P 37/02; A61P 35/00; C07D 295/00

(52) U.S. Cl. 514/232.8; 546/18; 514/278; 544/70

(57) ABSTRACT

The invention relates to new fredericamycin derivatives, to pharmaceutical drugs containing them or their salts, and to the use of the fredericamycin derivatives for the treatment of diseases, especially tumor diseases.

The invention relates to new fredericamycin derivatives, pharmaceuticals drugs containing them or their salts, and to the use of the fredericamycin derivatives for the treatment of diseases, especially tumor diseases.

Fredericamycin was isolated in 1981 from Streptomyces griseus and it exhibits anti-tumor activity.

Fredericamycin and several fredericamycin derivatives are known.

International Patent WO 2004/024696 describes an advantageous purification method for fredericamycin.

Heterocycles 37 (1994) 1893-1912, J. Am. Chem. Soc, 116 (1994) 9921-9926, J. Am. Chem. Soc. 116 (1994) 11275-11286, J. Am. Chem. Soc. 117 (1995) 11839-11849, JP 2000-072752 and J. Am. Chem. Soc. 123 (2001) all describe various, also enantioselective, total syntheses of fredericamycin A. J. Am. Chem. Soc. 127 (2005) 16442-16452 describes the biosynthesis path of fredericamycin A.

U.S. Pat. No. 4,673,768 describes alkali salts of fredericamycin A. U.S. Pat. No. 4,584,377 describes fredericamycin derivatives, especially derivatives acylated on rings A and B. U.S. Pat. No. 5,166,208 likewise describes fredericamycin derivatives, especially derivatives that have thio-substituents or amino-substituents on ring A. The derivatives are prepared semi-synthetically or totally synthetically. International Patent WO 03/080582 describes a plurality of fredericamycin derivatives that are derivatized on rings A, B, E and/or F. International Patent WO 03/087060 discloses other derivatives of fredericamycin, especially those in which ring E is further derivatized. International Patent WO 2004/004713 discloses other derivatives on rings A and B. There is a great need for additional fredericamycin derivatives that especially have modified profiles of action (side effects, etc.).

Surprisingly, it was found that fredericamycin derivatives that are derivatized especially on ring A or on rings A and E constitute potent pharmaceutical drugs. Moreover, a semi-synthetic possibility was found for introducing radicals to ring A or to both rings A and E, which make it possible to enhance the efficacy and, among other things, the water-solubility of the derivatives. Other ways for the derivatization that are known from the state of the art can also be carried outperformed on the derivatives according to the invention. Moreover, an alternative was found to make fredericamycin derivatives water-soluble by producing cyclodextrin inclusion compounds.

The invention relates to new fredericamycin derivatives having the general Formula Ia or Ib:

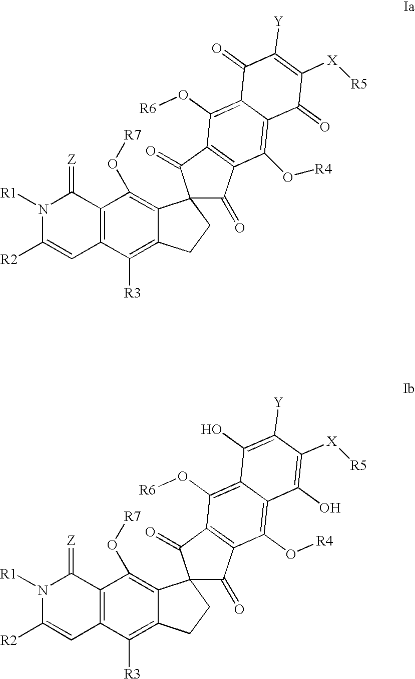

wherein
R1 stands for H, C₁-C₆-alkyl, cycloalkyl, C₁-C₄-alkyl-cycloalkyl,
R2 stands for H, C₁-C₁₄-alkyl, C₂-C₁₄-alkenyl, aryl, C₁-C₄-alkyl-aryl, heteroaryl, C₁-C₄-alkyl-heteroaryl, C₂-C₄-alkenyl-heteroaryl, cycloalkyl, C₁-C₄-alkyl-cycloalkyl, heterocycloalkyl, C₁-C₄-alkyl-heterocycloalkyl, CₘH₂ₘ₊ₒ₋ₚYₚ (with m=1 to 6, for o=1, p=1 to 2m+o; for m=2 to 6, o=−1, p=1 to 2m+o; for m=4 to 6, o=−2, p=1 to 2m+o; Y, independent of each other, is selected from the group consisting of halogen, OH, OR21, NH₂, NHR21, NR21R22, SH, SR21), (CH₂)ᵣCH₂NHCOR21, (CH₂)ᵣCH₂OCOR21, (CH₂)ᵣCH₂NHCSR21, (CH₂)ᵣCH₂S(O)nR21 with n=0, 1, 2, (CH₂)ᵣCH₂SCOR21, (CH₂)ᵣCH₂OSO₂—R21, (CH₂)ᵣCHO, CH₂—O—N═CH-aryl, CH₂—O—N═CH-hetaryl, CH₂—O—N═CH—R21, CH₂—O—N═CR21R22, CH₂—O—N═CH-cycloalkyl, CH═N—S-aryl, CH═N—S-hetaryl, (CH₂)ᵣCH═NOH, (CH₂)ᵣCH(OH)R21, —(CH₂)ᵣCH═NOR21, (CH₂)ᵣCH═NOCOR21, (CH₂)ᵣCH═NOCH₂CONR21R22, (CH₂)ᵣCH═NOCH(CH₃)CONR21R22, —(CH₂)ᵣCH═NOC(CH₃)₂CONR21R22, (CH₂)ᵣCH═N—NHCO—R23, (CH₂)ᵣCH═N—NHC(O)NH—R23, (CH₂)ᵣCH═N—NHC(S)NH—R23, (CH₂)ᵣCH═N—NHC(NH)NH—R23, (CH₂)ᵣCH═N—NHC(NH)—R23, (CH₂)ᵣCH═N—NHCO—CH₂NHCOR21, (CH₂)ᵣCH═N—O CH₂NHCOR21, (CH₂)ᵣCH═N—NHCS—R23, (CH₂)ᵣCH═CR24R25 (trans or cis), (CH₂)ᵣCOOH, (CH₂)ᵣCOOR21, (CH₂)ᵣCONR21R22, —(CH₂)ᵣCH═NR21, (CH₂)ᵣCH═N—NR21R22,

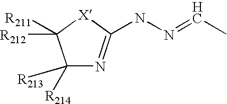

and the (CH₂)ᵣ-chain lengthened radical (CH₂)ᵣCH═N—N—(C₁-C₃-alkyl-NX′R211R212R213R214) (with X′═NR215, O, S and R211, R212, R213, R214, R215, independent of each other, stand for H or C₁-C₆-alkyl), —(CH₂)ᵣCH═N—NHSO₂-aryl, —(CH₂)ᵣCH═N—NHSO₂-heteroaryl, with r=0, 1, 2, 3, 4, 5, preferably 0,
R21, R22 independent of each other, stand for C₁-C₁₄-alkyl, C₁-C₁₄-alkanoyl, C₁-C₆-alkylhydroxy, C₁-C₆-alkylamino, C₁-C₆-alkylamino-C₁-C₆-alkyl, C₁-C₆-alkylamino-di-C₁-C₆-alkyl, cycloalkyl, C₁-C₄-alkylcycloalkyl, heterocycloalkyl, C₁-C₄-alkyl-heterocycloalkyl, aryl, aryloyl, C₁-C₄-alkyl-aryl, heteroaryl, heteroaryloyl, C₁-C₄-alkylheteroaryl, cycloalkanoyl, C₁-C₄-alkanoyl-cycloalkyl, heterocycloalkanoyl, C₁-C₄-alkanoyl-heterocycloalkyl, C₁-C₄-alkanoyl-aryl, C₁-C₄-alkanoyl-heteroaryl, mono- and di-sugar radicals that are linked via a C-atom that would carry an OH group in the sugar, whereby the sugars, independent of each other, are selected from the group consisting of glucuronic acid and its stereoisomers on all optical C-atoms, aldopentoses, aldohexoses, including their deoxy compounds (such as, for example, glucose, deoxyglucose, ribose, deoxyribose),
R23 independent of R21, has the same meanings as R21 or CH₂-pyridinium salts, CH₂-tri-C₁-C₆-alkyl ammonium salts,
R24 independent of R21, has the same meanings as R21 or H, CN, COCH₃, COOH, COOR21, CONR21R22, NH₂, NHCOR21
R25 independent of R21, has the same meanings as R21 or H, CN, COCH₃, COOH, COOR21, CONR21R22, NH₂, NHCOR21
R24, R25 together stand for C₄-C₈-cycloalkyl,
R3 stands for H, F, Cl, Br, I, OH, OR31, NO₂, NH₂, NHR31, NR31R32, NHCHO, NHCOR31, NHCOCF₃, CH₃₋ₘHalₘ (with Hal=Cl, F, especially F, and m=1, 2, 3), OCOR31, SCN, CN, N₃, CH₂NR331R332 (with R331, R332, which, independent of each other, can have the same meaning as R33), CH₂OH, CH₂OR33, CH₂SR33, C₂-C₁₄-alkyl, C₂-C₁₄-alkenyl, C₂-C₁₄-alkinyl, C₂-C₁₄-alkyl, C₂-C₁₄-alkenyl, C₂-C₁₄-alkinyl, aryl, C₁-C₄-alkyl-aryl, heteroaryl, C₁-C₄-alkyl-heteroaryl, whereby the aryls or heteroaryls can be substituted with another aryl, C₁-C₄-alkyl-aryl, O-aryl, C₁-C₄-alkyl-O-aryl, heteroaryl, C₁-C₄-alkyl-heteroaryl, O-heteroaryl or C₁-C₄-alkyl-O-heteroaryl; cycloalkyl, C₁-C₄-alkyl-cycloalkyl, heterocycloalkyl, C₁-C₄-alkyl-heterocycloalkyl, CₘH₂ₘ₊ₒ₋ₚYₚ (with m=2 to 6, for o=1, −1, p−1 to 2m+o; for m=4 to 6, o=3, p=1 to 2m+o; Y, independent of each other, is selected from the group consisting of halogen, OH, OR31, NH₂, NHR31, NR31R32, SH, SR31), CH₂NHCOR31, CH₂NHCSR31, CH₂S(O)nR31 with n=0, 1, 2, CH₂SCOR31, CH₂OSO₂—R31, CHO, CH═NOH, CH(OH)R31, —CH═NOR31, —CH═NOCOR31, —CH═NOCH₂CONR31R32, —CH═NOCH(CH₃)CONR31R32, —CH═NOC(CH₃)₂CONR31R32, —CH═N—NHCOR33, —CH═N—NHCO—CH₂NHCOR31, —CH═N—O—CH₂NHCOR31, —CH═N—NHCS—R33, —CH═CR34R35 (trans or cis), COOH, COOR31, CONR31R32, —CH═NR31, —CH═N—NR31R32,

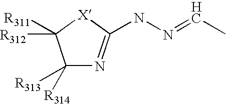

(with X′═NR315, O, S and R311, R312, R313, R314, R315, independent of each other, stand for H or C₁-C₆-alkyl), —CH═N—NHSO₂-aryl, —CH═N—NHSO₂-heteroaryl, and/or SCN, CN, N₃, CH₂NR331R332 (with R331, R332, which, independent of each other, can have the same meaning as R33), CH₂SR33,
R31, R32 independent of each other, stand for C₁-C₁₄-alkyl, C₁-C₁₄-alkanoyl, C₁-C₆-alkylhydroxy, C₁-C₆-alkylamino, C₁-C₆-alkylamino-C₁-C₆-alkyl, C₁-C₆-alkylamino-di-C₁-C₆-alkyl, cycloalkyl, C₁-C₄-alkylcycloalkyl, heterocycloalkyl, C₁-C₄-alkyl-heterocycloalkyl, aryl, aryloyl, C₁-C₄-alkyl-aryl, heteroaryl, heteroaryloyl, C₁-C₄-alkylheteroaryl, cycloalkanoyl, C₁-C₄-alkanoyl-cycloalkyl, heterocycloalkanoyl, C₁-C₄-alkanoyl-heterocycloalkyl, C₁-C₄-alkanoyl-aryl, C₁-C₄-alkanoyl-heteroaryl, mono- and di-sugar radicals that are linked via a C-atom that would carry an OH group in the sugar, whereby the sugars, independent of each other, are selected from the group consisting of glucuronic acid and its stereoisomers on all optical C-atoms, aldopentoses, aldohexoses including their deoxy compounds (such as, for example, glucose, deoxyglucose, ribose, deoxyribose),
R33 independent of R31, has the same meanings as R31 or CH₂-pyridinium salts, CH₂-tri-C₁-C₆-alkylammonium salts,
R34 independent of R31, has the same meanings as R31 or H, CN, COCH₃, COOH, COOR21, CONR31R32, NH₂, NHCOR31
R35 independent of R31, has the same meanings as R31 or H, CN, COCH₃, COOH, COOR31, CONR31R32, NH₂, NHCOR31
R34, R35 together stand for C₄-C₈-cycloalkyl,
R5 stands for H, C₁-C₆-alkyl, cycloalkyl, C₁-C₄-alkyl-cycloalkyl, heterocycloalkyl, C₁-C₄-alkyl-heterocycloalkyl, aryl, C₁-C₄-alkyl-aryl, heteroaryl, C₁-C₄-alkyl-heteroaryl,
R4, R6, R7 independent of each other, stand for H, C₁-C₆-alkyl, CO—R41
R41 independent of R21, has the same meanings as R21
X stands for O, S, NH, N—R8, whereby R8, independent of R5, can have the same meaning as R5, or R5 and R8, together with N, form a 4-, 5-, 6-, 7- or 8-membered heterocycloalkyl ring that can optionally contain another heteroatom selected from the group consisting of N, O, S,
or X—R5 together stand for H, F, Cl, Br, I, N₃
Y stands for F, Cl, Br, I, N₃, CN, CH₂NRY1RY2, CH₂OH, CH₂ORY1, CH₂SRY1, SCN, aryl, hetaryl (whereby RY1, RY2, independent of each other, can have the same meaning as R23), NRY1RY2, together with N, form a 4-, 5-, 6-, 7- or 8-membered heterocycloalkyl ring that can optionally contain another heteroatom selected from the group consisting of N, O, S and, in the case of X—R5 together standing for F, Cl, Br, I, N₃, then Y can also stand for H, W—R51, with W═O, S, NH, N—R81, R81 and R51, independent of each other, can have the same meaning as R5 or R51 and R81, together with N, form a 4-, 5-, 6-, 7- or 8-membered heterocycloalkyl ring that can optionally contain another heteroatom selected from the group consisting of N, O, S, and/or H, W—R51, with W═O, S, NH, N—R81, whereby R81 and R51, independent of each other, can have the same meaning as R5, or R51 and R81, together with N, form a 4-, 5-, 6-, 7- or 8-membered heterocycloalkyl ring that can optionally contain another heteroatom selected from the group consisting of N, O, S,
Z stands for O, S, NR9, whereby R9 can be H or C₁-C₆-alkyl,
   their stereoisomers, tautomers and their physiologically compatible salts or inclusion compounds.

Preference is given to compounds having Formula IIa or IIb

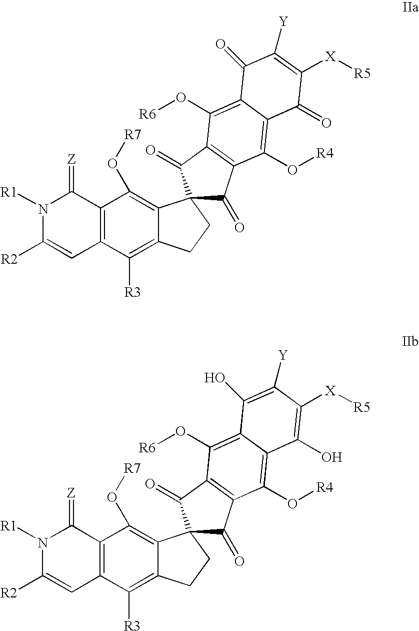

whereby the meaning of the radicals R, X, Y and Z is as given above, their tautomers and their physiologically compatible salts or inclusion compounds.

The invention also relates to compounds having Formulas Ia, Ib, IIa or IIb, in which the radicals R, aside from R3, have the meanings given above and R3, in comparison to when R3 equals H, increases the water-solubility—with the retention of all of the other radicals—by a factor of at least two, preferably by a factor of at least five, even more preferably by a factor of at least ten, especially preferably by a factor of at least fifty, especially by a factor of one hundred or even five hundred, The increase in the water-solubility is due, for example, to the introduction of groups that can form more hydrogen bridge compounds and/or that are polar and/or ionic. Preference is given to radicals R3 having greater water-solubility and the meaning given in the formulas.

The invention also relates to compounds having Formulas Ia, Ib, IIa or IIb, in which the radicals R, aside from R2, have the meanings given above and additionally R2, in comparison to when R2 equals CH═CH—CH═CH—CH₃, increases the water-solubility—with the retention of all of the other radicals—by a factor of at least two, preferably by a factor of at least five, even more preferably by a factor of at least ten, especially preferably by a factor of at least fifty, especially by a factor of one hundred or even five hundred. The increase in the water-solubility is due, for example, to the introduction of groups that can form more hydrogen bridge compounds and/or that are polar and/or ionic. Key intermediate products are compounds having an aldehyde function in R2. Preference is given to radicals R2 having greater water-solubility and the meaning given in the formulas. Especially preferred are derivatives with greater water-solubility in R2 and R3.

Preferred radicals R2 are heteroaryl, cycloalkyl, C₁-C₄-alkyl-cycloalkyl, heterocycloalkyl, C₁-C₄-alkyl-heterocycloalkyl, CₘH₂ₘ₊ₒ₋ₚYₚ (with m=1 to 6, for o=1, p=1 to 2m+o; for m=2 to 6, o=−1, p=1 to 2m+o; for m=4 to 6, o=−2, p=1 to 2m+o; Y, independent of each other, is selected from the group consisting of halogen, OH, OR21, NH₂, NHR21, NR21R22, SH, SR21), CH₂NHCOR21, CH₂NHCSR21, CH₂S(O)nR21 with n=0, 1, 2, CH₂SCOR21, CH₂OSO₂—R21, CH(OH)R21, —CH═NOCOR21, —CH═NOCH₂CONR21R22, —CH═NOCH(CH₃)CONR21R22, —CH═NOC(CH₃)₂CONR21R22, —CH═N—NHCO—R23, —CH═N—NHCO—CH₂NHCOR21, —CH═N—O—CH₂NHCOR21, —CH═N—NHCSR23, —CH═CR24R25 (trans or cis), CONR21R22, —CH═NR21,

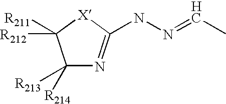

—CH═N—NR21R22, (with X′═NR215, O, S and R211, R212, R213, R214, R215, independent of each other, stand for H or C₁-C₆-alkyl), —CH═N—NHSO₂-aryl, —CH═N—NHSO₂-heteroaryl,

Preference is also given to compounds as indicated above, whereby the radicals R, preferably independent of each other, have one or more of the following meanings:
R1 stands for H, C₁-C₅-alkyl, cycloalkyl, especially H,
R2 stands for H, C₁-C₁₄-alkyl, C₂-C₁₄-alkenyl, aryl, C₁-C₄-alkyl-aryl, heteroaryl, C₁-C₄-alkyl-heteroaryl C₂-C₄-alkenyl-heteroaryl, cycloalkyl, C₁-C₄-alkyl-cycloalkyl, heterocycloalkyl, C₁-C₄-alkyl-heterocycloalkyl, CₘH₂ₘ₊ₒ₋ₚYₚ (with m=1 to 6, for o=1, p=1 to 2m+o; for m=2 to 6, o=−1, p=1 to 2m+o; for m=4 to 6, o=−2, p=1 to 2m+o; Y, independent of each other, is selected from the group consisting of halogen, OH, OR21, NH₂, NHR21, NR21R22, SH, SR21), (CH₂)ᵣCH₂NHCOR21, (CH₂)ᵣCH₂OCOR21, (CH₂)ᵣCH₂NHCSR21, (CH₂)ᵣCH₂S(O)nR21 with n=0, 1, 2, (CH₂)ᵣCH₂SCOR21, (CH₂)ᵣCH₂OSO₂—R21, (CH₂)ᵣCHO, CH₂—O—N═CH-aryl, CH₂—O—N═CH-hetaryl, CH₂—O—N═CH—R21, CH₂—O—N═CR21R22, CH₂—O—N═CH-cycloalkyl, CH═N—S-aryl, CH═N—S-hetaryl, (CH₂)ᵣCH═NOH, (CH₂)ᵣCH(OH)R21, —(CH₂)ᵣCH═NOR21, (CH₂)ᵣCH═NOCOR21, (CH₂)ᵣCH═NOCH₂CONR21R22, (CH₂)ᵣCH═NOCH(CH₃)CONR21R22, —(CH₂)ᵣCH═NOC(CH₃)₂CONR21R22, (CH₂)ᵣCH═N—NHCO—R23, (CH₂)ᵣCH═N—NHC(O)NH—R23, (CH₂)ᵣCH═N—NHC(S)NH—R23, (CH₂)ᵣCH═N—NHC(NH)NH—R23, (CH₂)ᵣCH═N—NHC(NH)—R23, (CH₂)ᵣCH═N—NHCO—CH₂NHCOR21, (CH₂)ᵣCH═N—O—CH₂NHCOR21, (CH₂)ᵣCH═N—NHCS—R23, (CH₂)ᵣCH═CR24R25 (trans or cis), (CH₂)ᵣCOOH, (CH₂)ᵣCOOR21, (CH₂)ᵣCONR21R22, —(CH₂)ᵣCH═NR21, (CH₂)ᵣCH═N—NR21R22,

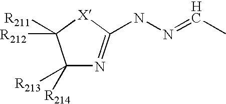

and the (CH₂)ᵣ-chain lengthened radical (CH₂)ᵣCH═N—N—(C₁-C₃-alkyl-NX′R211R212R213R214) (with X′═NR215, O, S and R211, R212, R213, R214, R215, independent of each other, stand for H or C₁-C₆-alkyl), —(CH₂)ᵣCH═N—NHSO₂-aryl, —(CH₂)ᵣCH═N—NHSO₂-heteroaryl, with r=0, 1, 2, 3, 4, 5, preferably 0, especially preferred are C₂-C₁₄-alkenyl, C₁-C₄-alkyl-heteroaryl, C₂-C₄-alkenyl-heteroaryl, CH═NOH, CH═NOR21,
R21, R22 independent of each other, stand for C₁-C₆-alkyl, cycloalkyl, aryl, C₁-C₄-alkyl-aryl, heteroaryl, C₁-C₄-alkyl-heteroaryl
R23 independent of R21, has the same meanings as R21 or CH₂-pyridinium salts, CH₂-tri-C₁-C₆-alkyl ammonium salts,
R24 independent of R21, has the same meanings as R21 or H, CN, COCH₃, COOH, COOR21, CONR21R22, NH₂, NHCOR21
R25 independent of R21, has the same meanings as R21 or H, CN, COCH₃, COOH, COOR21, CONR21R22, NH₂, NHCOR21
R24, R25 together stand for C₄-C₈-cycloalkyl,
R3 stands for H, F, Cl, Br, I, OH, OR31, NO₂, NH₂, NHR31, NR31R32, NHCHO, NHCOR31, NHCOCF₃, CH₃₋ₘHalₘ (with Hal=Cl, F, especially F, and m=1, 2, 3), OCOR31, SCN, CN, N₃, CH₂NR331R332 (with R331, R332, which, independent of each other, can have the same meaning as R33), CH₂OH, CH₂OR33, CH₂SR33, C₂-C₁₄-alkyl, C₂-C₁₄-alkenyl, C₂-C₁₄-alkinyl, C₂-C₁₄-alkyl, C₂-C₁₄-alkenyl, C₂-C₁₄-alkinyl, aryl, C₁-C₄-alkyl-aryl, heteroaryl, C₁-C₄-alkyl-heteroaryl, whereby the aryls or heteroaryls can be substituted with another aryl, C₁-C₄-alkyl-aryl, O-aryl, C₁-C₄-alkyl-O-aryl, heteroaryl, C₁-C₄-alkyl-heteroaryl, O-heteroaryl or C₁-C₄-alkyl-O-heteroaryl; cycloalkyl, C₁-C₄-alkyl-cycloalkyl, heterocycloalkyl, C₁-C₄-alkyl-heterocycloalkyl, CₘH₂ₘ₊ₒ₋ₚYₚ (with m=2 to 6, for o=1, −1, p=1 to 2m+o; for m=4 to 6, o=−3, p=1 to 2m+o; Y, independent of each other, is selected from the group consisting of halogen, OH, OR31, NH₂, NHR31, NR31R32, SH, SR31), CH₂NHCOR31, CH₂NHCSR31, CH₂S(O)nR31 with n=0, 1, 2, CH₂SCOR31, CH₂OSO₂—R31, CHO, CH═NOH, CH(OH)R31, —CH—NOR31, —CH═NOCOR31, —CH═NOCH₂CONR31R32, —CH—NOCH(CH₃)CONR31R32, —CH═NOC(CH₃)₂CONR31R32, —CH═N—NHCOR33, —CH═N—NHCO—CH₂NHCOR31, —CH═N—O—CH₂NHCOR31, —CH═N—NHCS—R33, —CH═CR34R35 (trans or cis), COOH, COOR31, CONR31R32, —CH═NR31, —CH—N—NR31R32,

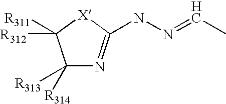

(with X′═NR315, O, S and R311, R312, R313, R314, R315, independent of each other, stand for H or C₁-C₆-alkyl), —CH═N—NHSO₂-aryl, —CH═N—NHSO₂-heteroaryl, especially preferred are H, F, Cl, Br, I, NR31R32, especially Br, I, and/or CH₂NR331R332 (with R331, R332, which, independent of each other, can have the same meaning as R33),
R331, R332 independent of each other, stand for C₁-C₄-alkyl,
R31, R32 independent of each other, stand for C₁-C₄-alkyl,
R5 stands for H, C₁-C₃-alkyl, cycloalkyl, heterocycloalkyl,
R4, R6, R7 independent of each other, stand for H, C₁-C₅-alkyl, CO—R41, especially in each case H,
R41 independent of R21, has the same meanings as R21
X stands for O, S, NH, N—R8, especially preferably for O, NH, N—R8, whereby R8 can have the same meaning as R5 and in the case of N—R8, especially preferably R5 and R8, together with N, form a 6-membered heterocycloalkyl ring that can optionally contain another heteroatom selected from the group consisting of N, O, and is especially piperazino or morpholino, especially preferably for O, NH,
or X—R5 together stand for H,
Y stands for H, F, Cl, Br, I, N₃, especially for Br, I
Z stands for O, S, NH, especially for O
   their stereoisomers, tautomers and their physiologically compatible salts or inclusion compounds.

Moreover, it is preferred that if

R3=SCN, CN, N₃, CH₂NR331R332 (with R331, R332, which, independent of each other, can have the same meaning as R33), is CH₂SR33,
then Y═H, W—R51, with W═O, S, NH, N—R81, whereby R81 and R51, independent of each other, can have the same meaning as R5, or R51 and R81 together with N, form a 4-, 5-, 6-, 7- or 8-membered heterocycloalkyl ring that can optionally contain another heteroatom selected from the group consisting of N, O, S, and if R3=H, F, Cl, Br, I, OH, OR31, NO₂, NH₂, NHR31, NR31R32, NHCHO, NHCOR31, NHCOCF₃, CH₃₋ₘHalₘ (with Hal=Cl, F, especially F, and m=1, 2, 3), OCOR31, SCN, CN, N₃, CH₂NR331R332 (with R331, R332, which, independent of each other, can have the same meaning as R33), CH₂OH, CH₂OR33, CH₂SR33, C₂-C₁₄-alkyl, C₂-C₁₄-alkenyl, C₂-C₁₄-alkinyl, C₂-C₁₄-alkyl, C₂-C₁₄-alkenyl, C₂-C₁₄-alkinyl, aryl, C₁-C₄-alkyl-aryl, heteroaryl, C₁-C₄-alkyl-heteroaryl, whereby the aryls or heteroaryls can be substituted with another aryl, C₁-C₄-alkyl-aryl, O-aryl, C₁-C₄-alkyl-O-aryl, heteroaryl, C₁-C₄-alkyl-heteroaryl, O-heteroaryl or C₁-C₄-alkyl-O-heteroaryl; cycloalkyl, C₁-C₄-alkyl-cycloalkyl, heterocycloalkyl, C₁-C₄-alkyl-heterocycloalkyl, CₘH₂ₘ₊ₒ₋ₚYₚ (with m=2 to 6, for o=1, −1, p=1 to 2m+o; for m=4 to 6, o=−3, p=1 to 2m+o; Y, independent of each other, is selected from the group consisting of halogen, OH, OR31, NH₂, NHR31, NR31R32, SH, SR31), CH₂NHCOR31, CH₂NHCSR31, CH₂S(O)nR31 with n=0, 1, 2, CH₂SCOR31, CH₂OSO₂—R31, CHO, CH═NOH, CH(OH)R31, —CH═NOR31, —CH═NOCOR31, —CH═NOCH₂CONR31R32, —CH═NOCH(CH₃)CONR31R32, —CH═NOC(CH₃)₂CONR31R32, —CH═N—NHCOR33, —CH═N—NHCO—CH₂NHCOR31-CH═N—O—CH₂NHCOR31, —CH═N—NHCSR33, —CH═CR34R35 (trans or cis), COOH, COOR31, CONR31R32, —CH═NR31, —CH═N—NR31R32,

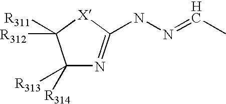

(with X′═NR315, O, S and R311, R312, R313, R314, R315, independent of each other, stand for H or C₁-C₆-alkyl), —CH═N—NHSO₂-aryl, —CH═N—NHSO₂-heteroaryl, Y═F, CI, Br, I, N₃, CN, CH₂NRY1RY2, CH₂OH, CH₂ORY₁, CH₂SRY1, SCN, aryl, hetaryl (whereby RY1, RY2, independent of each other, can have the same meaning as R23), NRY1RY2, together with N, form a 4-, 5-, 6-, 7- or 8-membered heterocycloalkyl ring that can optionally contain another heteroatom selected from the group consisting of N, O, S, and in the case of X—R5 together stand for F, Cl, Br, I, N₃, Y can also be H, W—R51, with W O, S, NH, N—R81, R81 and R51, independent of each other, can have the same meaning as R5, or R51 and R81 together with N, form a 4-, 5-, 6-, 7- or 8-membered heterocycloalkyl ring that can optionally contain another heteroatom selected from the group consisting of N, O, S.

Special preference is given to compounds, their stereoisomers, tautomers and their physiologically compatible salts or inclusion compounds, selected from the group consisting of the compounds of the examples as well as of the compounds that have combinations of the various substituents of the compounds of these examples.

Moreover, preference is given to pharmaceutical drugs containing the above-mentioned compounds having Formula I or II, along with the customary carriers and auxiliaries.

The above-mentioned pharmaceutical drugs in combination with other active ingredients are also preferred for the treatment of tumors.

These compounds according to the invention are used for the production of pharmaceutical drugs for treating tumors, especially those that can be treated through the inhibition of topoisomerases I and/or II. Tumors that can be treated with the substances according to the invention are, for example, leukemia, lung cancer, melanomas, prostate tumors and colon tumors. The compounds according to the invention are also used for the production of pharmaceutical drugs for treating tumors that can be treated through the inhibition of the peptidyl-prolyl isomerase PIN-1. Such tumors are especially prostate tumors and breast cancer.

Moreover, the compounds according to the invention can be used for the production of pharmaceutical drugs for treating neurodermatitis, parasites and for immunosuppression.

In the description and in the claims, the following definitions apply to the individual substituents:

The term “alkyl” on its own or as part of another substituent means a linear or branched alkyl chain radical of the length indicated in each case and optionally a CH₂-group that can be substituted by a carbonyl function. Thus, for example, C₁₋₄-alkyl means methyl, ethyl, 1-propyl, 2-propyl, 2-methyl-2-propyl, 2-methyl-1-propyl, 1-butyl, 2-butyl, C₁₋₆-alkyl, for example, C₁₋₄-alkyl, pentyl, 1-pentyl, 2-pentyl, 3-pentyl, 1-hexyl, 2-hexyl, 3-hexyl, 4-methyl-1-pentyl or 3,3-dimethyl-butyl.

The term “C₁₋₆-alkylhydroxy” on its own or as part of another substituent means a linear or branched alkyl chain radical of the length indicated in each case that can be saturated or unsaturated and that carries an OH group such as, for example, hydroxymethyl, hydroxyethyl, 1-hydroxypropyl, 2-hydroxypropyl.

The term “alkenyl” on its own or as part of another substituent means a linear or branched alkyl chain radical having one or more C═C double bonds of the length indicated in each case, whereby several double bonds are preferably conjugated. Thus, for example, C₂₋₆-alkenyl means ethenyl, 1-propenyl, 2-propenyl, 2-methyl-2-propenyl, 2-methyl-1-propenyl, 1-butenyl, 2-butenyl, 1,3-butadienyl, 2,4-butadienyl, 1-pentenyl, 2-pentenyl, 3-pentenyl, 1,3-pentadienyl, 2,4-pentadienyl, 1,4-pentadienyl, 1-hexenyl, 2-hexenyl, 1,3-hediexyl, 4-methyl-1-pentenyl or 3,3-dimethyl-butenyl.

The term “alkinyl” on its own or as part of another substituent means a linear or branched alkyl chain radical having one or more CC triple bonds of the length indicated in each case, whereby additional double bonds can also be present. Thus, for example, C₂₋₆-alkinyl means ethinyl, 1-propinyl, 2-propinyl, 2-methyl-2-propinyl, 2-methyl-1-propinyl, 1-butinyl, 2-butinyl, 1-pentinyl, 2-pentinyl, 3-pentinyl, 1,4-pentadiinyl, 1-pentin-4-enyl, 1-hexinyl, 2-hexinyl, 1,3-hexdiinyl, 4-methyl-1-pentinyl or 3,3-dimethyl-butinyl.

The term “halogen” stands for fluorine, chlorine, bromine, iodine, preferably for bromine and chlorine.

The term “NR21R22” or analogous NRx1Rx2 also stand for a dialkylamino group, whereby the two alkyl groups, together with N, can also form a 5- or 6-membered ring.

The term “cycloalkyl” on its own or as part of another substituent encompasses saturated, cyclic hydrocarbon groups having 3 to 8 C-atoms such as, for example, cyclopropyl, cyclobutyl, cyclopentyl, cyclohexyl, 4-methyl-cyclohexyl, cyclohexylmethylene, cycloheptyl or cyclooctyl.

The term “heterocycloalkyl” on its own or as part of another substituent comprises cycloalkyl groups, wherein up to two 0H₂ groups can be substituted by oxygen, sulfur or nitrogen atoms and another CH₂ group can be substituted by a carbonyl function such as, for example, pyrrolidine, piperidine, morpholine or

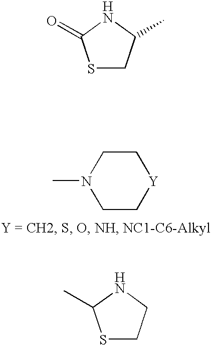

The term “aryl” on its own or as part of another substituent encompasses aromatic ring systems with up to 3 rings, in which at least one ring system is aromatic and having up to 3 substituents, preferably up to 1 substituent, whereby the substituents, independent of each other, have the meaning C₁-C₆-alkyl, OH, NO₂, CN, CF₃, OR11, SH, SR11, C₁-C₆-alkylhydroxy, C₁-C₆-alkyl-OR11, COOH, COOR11, CONH₂, CONR11R12, CHO, CH═NO—C₁-C₁₀-alkyl, C₁-C₁₀-alk-1-enyl, NH₂, NHR11, NR11R12, halogen, whereby the radicals R11, R12, independent of each other, can mean C₁-C₁₀-alkyl, cycloalkyl, C₁-C₄-alkylcycloalkyl.

Preferred aryls, in addition to phenyl and 1-naphthyl and 2-naphthyl are:

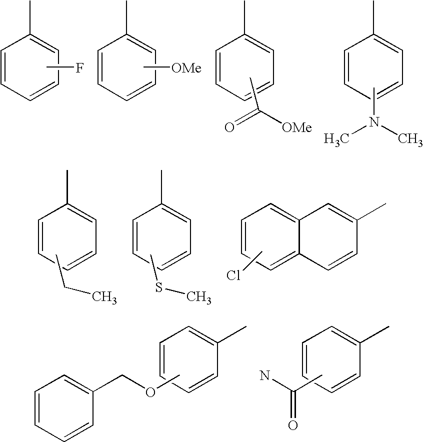

The term “heteroaryl” on its own or as part of another substituent encompasses aromatic ring systems with up to 3 rings and up to 3 of the same or different heteroatoms N, S, O in which at least 1 rings is aromatic and having up to 3 substituents, preferably up to 1 substituent, whereby the substituents, independent of each other, have the meaning C₁-C₆-alkyl, OH, NO₂, CN, CF₃, OR11, SH, SR11, C₁-C₆-alkylhydroxy, C₁-C₆-alkyl-OR11, COOH, COOR11, CONH₂, CONR11R12, CHO, CH═NO—C₁-C₁₀-alkyl, C₁-C₁₀-alk-1-enyl, NH₂, NHR11, NR11R12, halogen, whereby the radicals R11, R12, independent of each other, can mean C₁-C₁₀-alkyl, cycloalkyl, C₁-C₄-alkyl-cycloalkyl.

Preferred heteroaryls are:

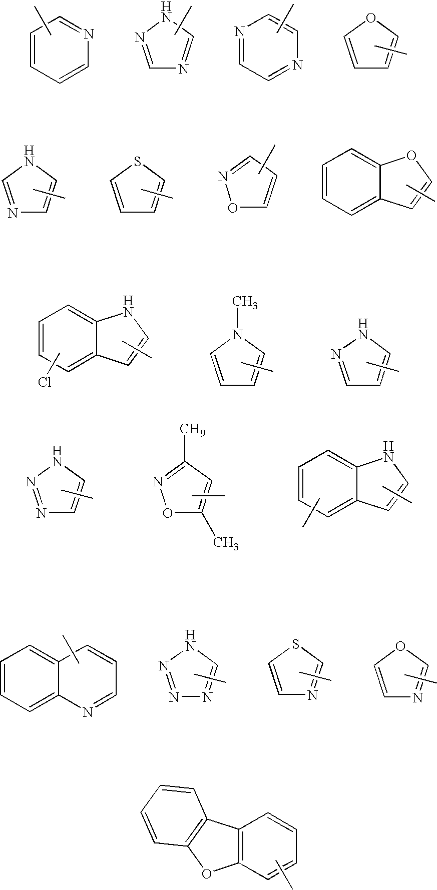

Special preference is given to 2-furyl, 3-furyl, 2-thiophenyl, 3-thiophenyl, 3-pyridinyl, 4-pyridinyl, 4-isoxazolyl, 2-N-methylpyrrolyl, and 2-pyrazinyl. These are especially preferred as radical R3.

The term “ring system” generally refers to 3-, 4-, 5-, 6-, 7-, 8-, 9- or 10-membered rings. Preference is given to 5- and 6-membered rings. Moreover, ring systems with one or two anellated rings are preferred.

The compounds having Formula I can be used as such or, if they have acidic or basic groups, in the form of their salts with physiologically compatible bases or acids. Examples of such acids are: hydrochloric acid, citric acid, trifluoroacetic acid, tartaric acid, lactic acid, phosphoric acid, methane sulfonic acid, acetic acid, formic acid, maleic acid, fumaric acid, succinic acid, hydroxy succinic acid, sulfuric acid, glutaric acid, asparaginic acid, pyruvic acid, benzoic acid, glucuronic acid, oxalic acid, ascorbic acid and acetyl glycine. Examples of bases are alkali ions, preferably Na, K, earth alkali ions, preferably Ca, Mg, ammonium ions.

The compounds according to the invention can be administered orally in the usual manner. They can also be administered intravenously, intramuscularly, with vapors or sprays through the nasopharyngeal space.

The dosage depends on the age, condition and weight of the patient as well as on the mode of administration. As a rule, the daily does of active ingredient per person lies between about 0.1 μg/kg and 1 g/kg in the case of oral administration. This dose can be administered in 2 to 4 individual doses or once per day in a slow-release form.

The new compounds can be used in the usual galenic administration form as a solid or a liquid, for example, as tablets, film tablets, capsules, powders, granulates, coated tablets, solutions or sprays. They are manufactured in the usual manner. The active ingredients can be processed with the usual galenic auxiliaries such as tablet binders, fillers, preservatives, tablet disintegrants, flow regulators, softeners, wetting agents, dispersants, emulsifiers, solvents, retardants, antioxidants and/or propellant gases (see H. Sucker et al.: Pharmazeutische Technologie [Pharmaceutical Technology], published by Thieme-Verlag, Stuttgart, Germany, 1978). The administration forms thus obtained normally contain the active ingredient in an amount of 0.1% to 99% by weight.

### Experimental Part

Fredericamycin A can be obtained through fermentation or totally synthetically using generally known methods. The fredericamycin derivatives according to the invention can be made either from fredericamycin A or from known fredericamycin derivatives using the indicated methods directly or by varying the indicated methods. The reduced forms of Formulas Ib and IIb can be created by mild reducing agents from the corresponding compounds having Formulas Ia and IIa.

### Preparation of the Substances

Fredericamycin (1) or fredericamycin derivatives—using halogenation agents such as N-chlorosuccinimide (NCS), bromosuccinimide (NBS), N-iodosuccinimide (NIS), fluorination agents such as Selectfluor® or elementary Br₂, Cl₂, interhalogen compounds—can be reacted at good yields to form the corresponding halogenated fredericamycin derivatives (Schema 1). The amination and subsequent second halogenation results in bis-halogenated fredericamycin derivatives with different substitution patterns (Schema 2).

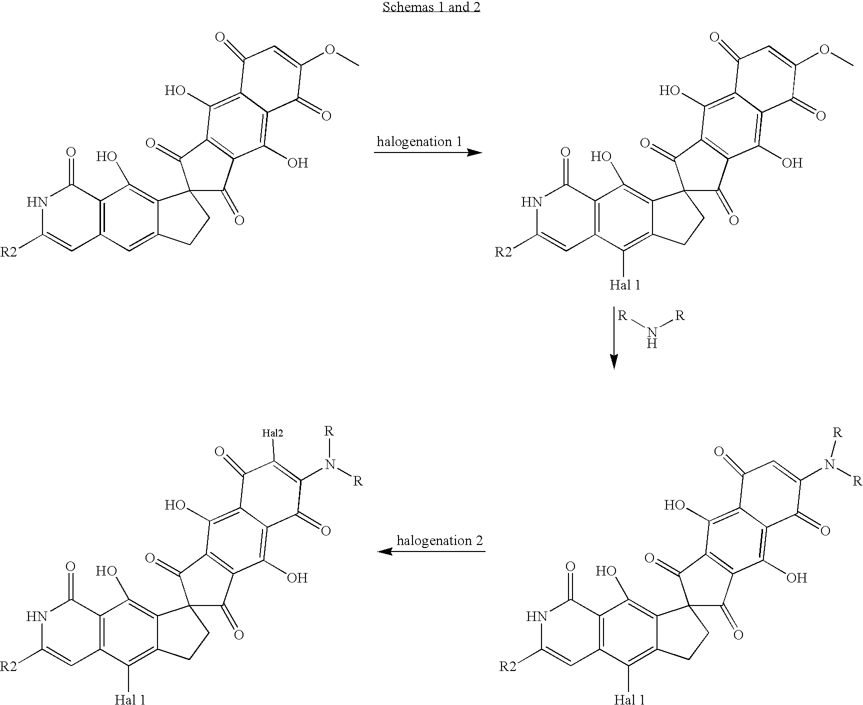

Hal2, independent of Hal1: halogen

For the synthesis of other water-soluble fredericamycin derivatives, fredericamycin (1) was first hydroxylated with osmium(IV)oxide on the diene side chain (see Schema 3).

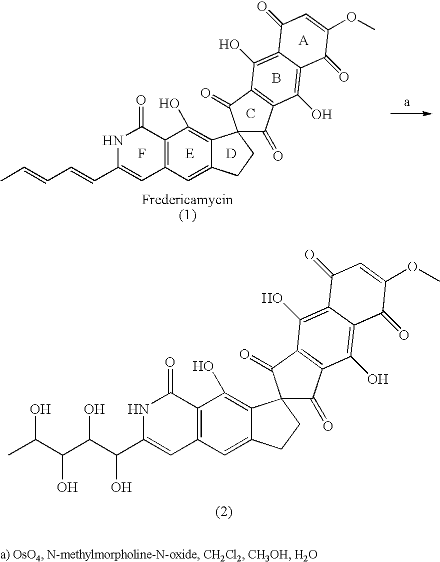

Fredericamycin-tetrol (2) likewise serves as an important intermediate stage for the synthesis of the fredericamycin derivatives cited in this patent and having a high solubility and/or activity profile. Through iodate cleavage with sodium metaperiodate or carrier-bound periodate, the tetrol side chain can be degraded to form fredericamycin aldehyde (3) in very high yields (see Schema 4).

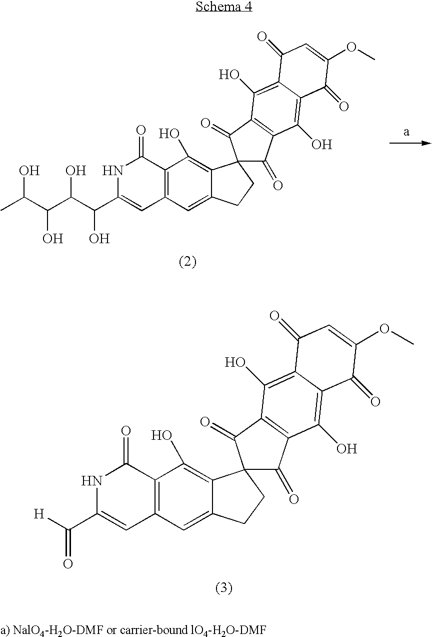

This aldehyde can be reacted, for example, by means of bromination reagents such as N-bromosuccinimide, bromine or other bromine-generating reagents (or other halogenation reagents) to form the nucleus-brominated compound (4) or the nucleus-halogenated compound (see Schema 5).

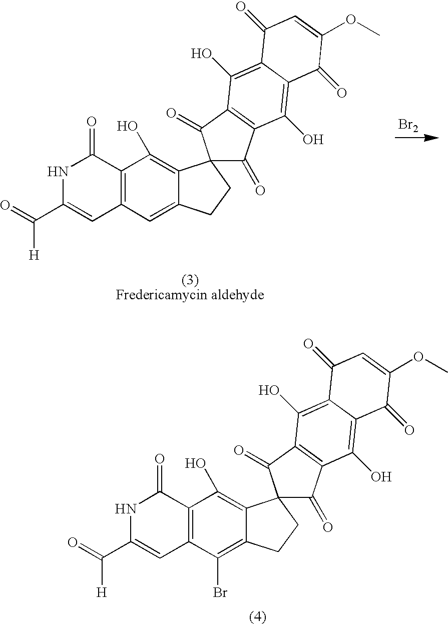

As an example of a substance library, the aldehyde (3) can be reacted, for example, with hydroxylamines and hydrazines to form the corresponding R3-substituted oximes. Amino exchange, nucleophilic substitution or C—C bonds are shown in Schema 6.

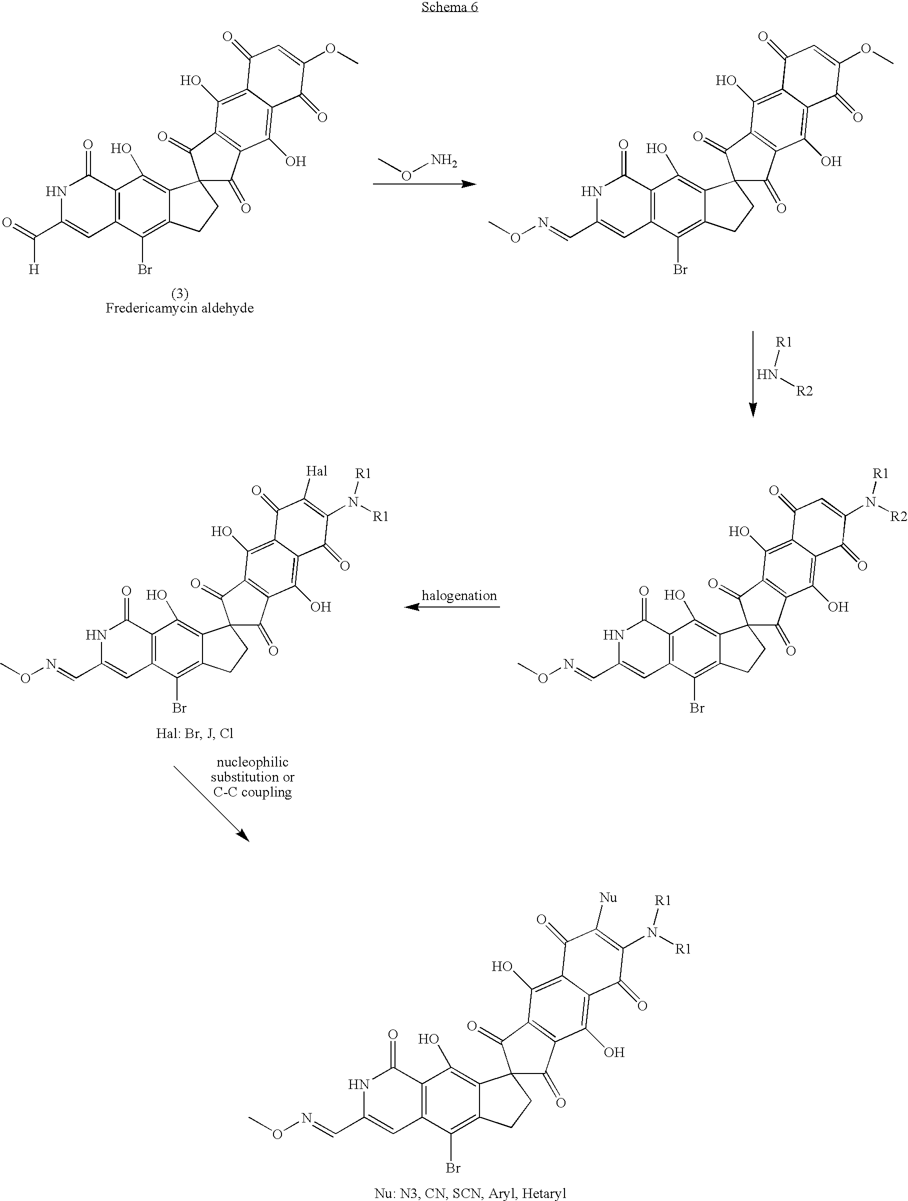

The following schemas show—on the basis of fredericamycin and its derivatives—how one can analogously obtain derivatives according to the invention.

Electrophilic substitution on the E ring and exchange of the methoxy group on the A ring

1)

Fredericamycin and its side-chain substituted derivatives can be amino-methylated under anhydrous conditions on the E ring with dimethylmethylene ammoniumohloride (Mannich salt) known from the literature.

The exchange of the methoxy grouping on the A-ring of the fredericamycin as well as on the derivatives is possible using primary, secondary or aromatic amines. Here, the components are stirred with the corresponding primary or secondary amines at room temperature in DMF or in another inert solvent. In the case of aromatic amines, catalysis with Lewis acids such as tin(IV)chloride, etc. is necessary. Halogenation with NBS or bromine supplies the F-ring halogenated derivatives (see Schema 7).

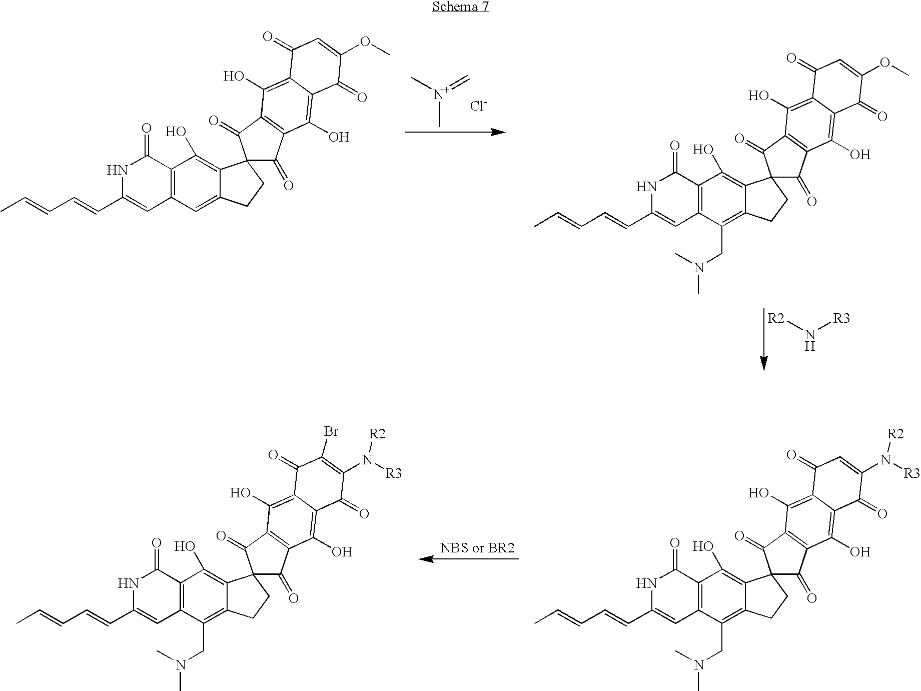

If the Mannich reaction is carried out with aqueous formaldehyde and amine on the demethylated fredericamycin, then the aminomethylation takes place on the A ring. The OH function on the A ring can be converted via the triflate into the amino compound or alkoxy compound (see Schema 8).

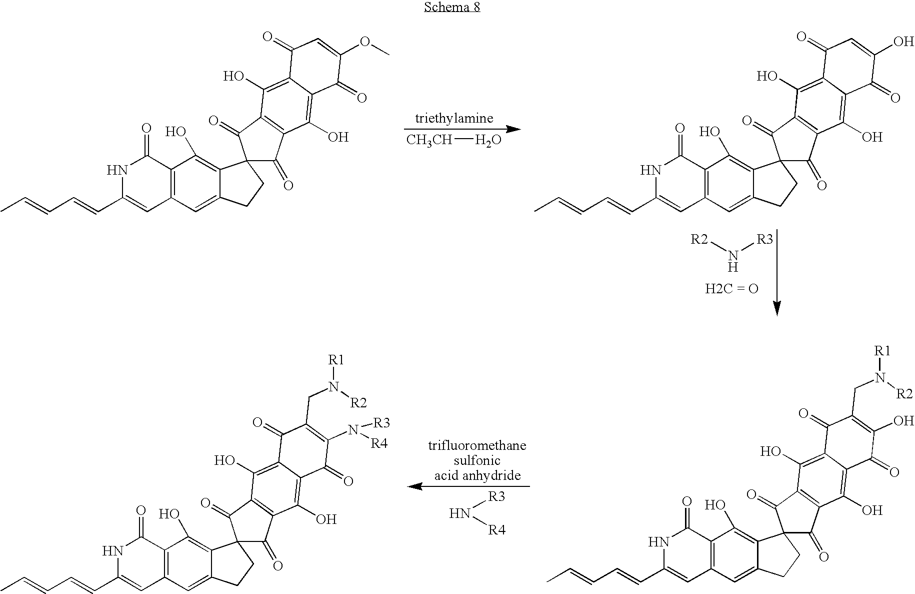

Fredericamycin or fredericamycin derivatives can be electrophilically substituted on the E ring with dirhodane produced in situ (Schema 9).

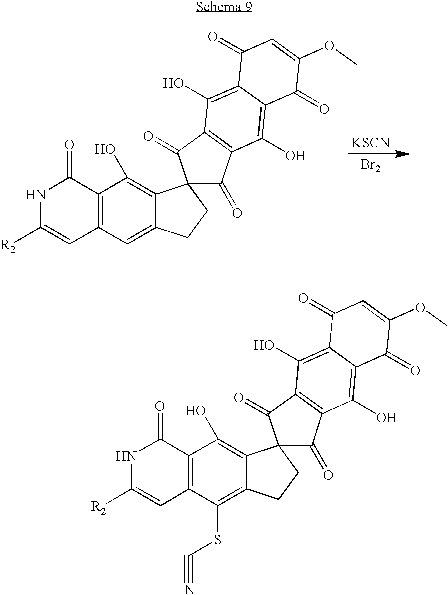

### EXAMPLES

### Example 1

### (8S)-4′,9,9′-trihydroxy-5,7′-dibromo-6′-dimethylamino-1,1′,3′,5′,8′-pentaoxo-1,1′,2,3′,5′,6,7,8′-octahydrospiro-[cyclopenta[g]-isoquinoline-8,2′-cyclopenta[b]naphthalene]-3-carbaldehyde (Compound 1)

5.0 mg (8.4 μmol) of bromine dimethylamino-fredericamycin aldehyde are dissolved under N₂ in 1 ml of dry dimethylformamide. 3.0 mg (16.9 μmol) of N-bromosuccinimide are added at room temperature and stirred at room temperature. After 90 minutes, this mixture is diluted with 15 ml of water and the precipitated sediment is aspirated. The residue dried in a vacuum is picked up in 25 ml of dichloromethane, washed with water and concentrated after being dried over sodium sulfate.

Yield: 3.5 mg (62% of the theoretical yield) of a red crystal powder: M/e=673, λₘₐₓ=507.0 nm

### Example 2

### (8S)-4′,9,9′-trihydroxy-5,7′-dibromo-6′-dimethylamino-1,1′,3′,5′,8′-pentaoxo-111′,2,3′,5′,6,7,8′-otahydrospiro-[cyclopenta[g]-isoquinoline-8,2′-cyclopenta[b]naphthalene]-3-carbaldehyde-O-isopropyloxime (Compound 2)

83.0 mg (128.0 μmol) of bromine dimethylamino-fredericamycin aldehyde-O-isopropyloxime are dissolved under N₂ in 2 ml of absolute dimethylformamide. 128 μl of a 0.1 M bromine solution in DMF are added at room temperature. After 1 hour, the mixture is added to 40 ml of water. The precipitated residue is aspirated and subsequently washed with methanol. After purification over Sephadex® LH-20 with dichloromethane/methanol/trifluoroacetic acid 30/20/0.1, one obtains 42.0 mg (45% of the theoretical yield) of a red solid. M/e=730.0; λₘₐₓ=504.0 nm.

### Example 3

### (8S)-4′,9,9′-trihydroxy-5,7′-dibromo-6′-methylamino-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 3)

53.8 mg (100 μmol) of methylamino-fredericamycin are dissolved under N₂ in 2 ml of absolute dimethylformamide. 200 μl of a 0.2M solution of N-bromosuccinimide in DMF are added at room temperature. After 16 hours, the solvent is aspirated in a high vacuum. The residue is purified over Sephadex® LH-20 with dichloromethane/methanol/trifluoroacetic acid 30/20/0.1.

Yield: 52.0 mg (75% of the theoretical yield) of a red solid. M/e=696.0; λmax=506.0 nm.

### Example 4

### (BS)-4′,9,9′-trihydroxy-5,7′-dibromo-6′-morpholino-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 5)

59.5 mg (100 μmol) of morpholino-fredericamycin are dissolved under N₂ in 2 ml of absolute dimethylformamide. 200 μl of a 0.2M solution of N-bromosuccinimide in DMF are added at room temperature. After 3 hours, another 200 μl of a 0.2M NBS solution are added and this is stirred for another hour. The solvent is aspirated in a high vacuum and the residue is purified over Sephadex® LH-20 with dichloromethane/methanol/trifluoroacetic acid 30/20/0.1. Purification is carried out once again using preparative HPLC RP18 with acetonitrile/water.

Yield: 23.0 mg (31% of the theoretical yield) of a red solid. M/e=753.0; λₘₐₓ=500.0 nm.

### Example 5

### (8S)-4′,9,9′-trihydroxy-5,7′-dibromo-6′-dimethylamino-1,1′,3′,5′,8′-pentaoxo-1,1′,2,3′,5′,6,7,8′-octahydrospiro-[cyclopenta[g]-isoquinoline-8,2′-cyclopenta[b]naphthalene]-3-carbaldehyde-O-methyloxime (Compound 9)

50.0 mg (80.3 μmol) of bromodimethylamino-fredericamycin aldehyde-O-methyloxime are dissolved under N₂ in 5 ml of absolute dimethylformamide. 14.3 mg (80.3 μmol) of N-bromosuccinimide in 1 ml of DMF are added at room temperature. After the mixture is stirred at room temperature for 3 hours, the solvent is aspirated in a high vacuum and the residue is purified over Sephadex® LH-20 with dichloromethane/methanol/trifluoroacetic acid 80/10/0.1.

Yield: 47.0 mg (83% of the theoretical yield) of a red solid. M/e=702.0; λₘₐₓ=504.0 nm.

### Example 6

### (8S)-4′,9,9′-trihydroxy-5,7′-dibromo-6-cyclopropylamino-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 10)

56.5 mg (100.0 μmol) of cyclopropylamino-fredericamycin are dissolved under N₂ in 5 ml of absolute dimethylformamide. 36.0 mg (202.2 μmol) of N-bromosuccinimide dissolved in 2 ml of DMF are added at room temperature.

After 2 hours of stirring at room temperature, the solvent is aspirated in a high vacuum and the residue is purified over Sephadex® LH-20 with dichloromethane/methanol/trifluoroacetic acid 80/10/0.1.

Yield: 38.0 mg (52% of the theoretical yield) of a red solid. M/e=723.0; λₘₐₓ=504.0 nm.

### Example 7

### (8S)-4,9,9′-trihydroxy-5,7′-dibromo-6′-cyclopropylamino-1,1′,3′,5′,8′-pentaoxo-1,1′,2,3′,5′,6,7,8′-octahydrospiro-[cyclopenta[g]-isoquinoline-8,2′-cyclopenta[b]naphthalene]-3-carbaldehyde-O-methyloxime (Compound 12)

60.0 mg (108.0 μmol) of cyclopropylamino-fredericamycin aldehyde methoxime are dissolved under N₂ in 5 ml of absolute dimethylformamide. 40.3 mg (226.8 μmol) of N-bromosuccinimide are added at room temperature. After the mixture is stirred at room temperature for 2 hours, the solvent is aspirated in a high vacuum and the residue is purified over Sephadex® LH-20 with dichloromethane/methanol/trifluoroacetic acid 80/10/0.1.

Yield: 28.0 mg (36% of the theoretical yield) of a red solid. M/e=714.0; λₘₐₓ=500.0 nm.

### Example 8

### (8S)-4′,9,9-trihydroxy-5,7′-dibromo-6′-cyclopropylamino-3-[(1E,3E)-penta-1,3-dienyl]-6,7-dihydrospiro[cyclopenta[g]-isoquinoline-8,22-cyclopenta[b]-naphthalene]-1,1′-3′,5′,8′(2H)-pentone (Compound 15)

10.0 mg (15.4 μmol) of 2-fluoroethylamino bromo-fredericamycin are dissolved under N₂ in 1 ml of absolute dimethylformamide. 2.7 mg (15.4 μmol) of N-bromosuccinimide are added at room temperature. After the mixture is stirred at room temperature for 5 hours, 100 ml of water/1% trifluoroacetic acid are added. The precipitate is aspirated and washed with water.

Yield: 4.0 mg (36% of the theoretical yield) of a red solid. M/e=729.0; λₘₐₓ=504.0 nm.

### Example 9

### (8S)-4′,9,9′-trihydroxy-7′-piperidinomethyl-6′-hydroxy-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 16)

20.0 mg (38.1 μmol) of hydroxy fredericamycin (demethylated fredericamycin) are placed under N₂ into 4 ml of ethanol. After the addition of 4.0 μl (40.3 μmol) of piperidine and 3.2 μl (115.0 μmol) of a 37%-aqueous solution of formaldehyde, the mixture is stirred at room temperature for 30 minutes. It is then heated to reflux temperature for 3 hours. The mixture is added to 80 ml of water (with 1% trifluoroacetic acid). This is followed by aspiration and drying in a vacuum.

Yield: 23.0 mg (97% of the theoretical yield) of a red solid. M/e=623.0; λₘₐₓ=500.0 nm.

### Example 10

### (8S)-4′,9,9′-trihydroxy-7′-dimethylaminomethyl-6′-hydroxy-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 18)

200.0 mg (381.0 μmol) of hydroxy fredericamycin (demethylated fredericamycin) are placed under N₂ into 40 ml of ethanol. After the addition of 286.0 μl (571.5 μmol) of dimethylamine (2M in methanol) and 57.0 μl (762.0 μmol) of a 37%-aqueous solution of formaldehyde, the mixture is stirred at room temperature for 30 minutes. It is then heated to 60° C. [140° F.] for 7 hours. Subsequently, the mixture is added to 300 ml of cold water (with 1% trifluoroacetic acid). This is followed by aspiration and drying in a vacuum.

Yield: 193.0 mg (87% of the theoretical yield) of a red solid. M/e=583.0; λₘₐₓ=504.0 nm.

### Example 11

### 1-deoxy-5-C-[(8R)-4′,9,9′-trihydroxy-6′-hydroxy-7′-dimethylamino-1,1′,3′,5′,8′-pentaoxo-1,1′,2,3′,5′,6,7,8′-octahydrospiro[cyclopenta[g]-isoquinoline-8,2′-cyclopenta[b]-naphthalen-3-yl]-pentitol (Compound 20)

22.5 mg (38.0 μmol) of hydroxy fredericamycin tetrol are placed under N₂ into 6 ml of ethanol, After the addition of 20.0 μl (40.0 μmol) dimethylamine solution (2M in methanol) and 3.2 μl (115.0 μmol) of a 37%-aqueous solution of formaldehyde, the mixture is stirred at room temperature for 30 minutes. It is then heated for 26 hours to 60° C. [140° F.]. After cooling off, the mixture is added to 100 ml of water (with 1% trifluoroacetic acid). This is followed by aspiration and drying in a vacuum.

Yield: 21.0 mg (96% of the theoretical yield) of a red solid. M/e=651.0; λₘₐₓ=498.0 nm.

### Example 12

### (8S)-4′,9,9′-trihydroxy-5,7′-diiodo-6′-methylamino-3-[(1E,3E)-penta-1,3-dienyl-6,7-dihydrospiro[cyclopenta[g]-isoquinoline-8,2′-cyclopenta[b]-naphthalene]-1,1′-3′,5′,8′(2H)-pentone (Compound 24)

50.0 mg (92.8 μmol) of methylamino-fredericamycin are dissolved under N₂ in 5 ml of absolute dimethylformamide. 48.8 mg (218.5 μmol) of N-iodosuccinimide are added at room temperature. After the mixture is stirred at room temperature for 5 hours, 100 ml of water/1% trifluoroacetic acid are added. The precipitate is aspirated and washed with water.

Yield: 7.2 mg (10% of the theoretical yield) of a red solid. M/e=791.0; λₘₐₓ=506.0 nm.

Compounds 4, 6-8, 11, 13, 14, 17, 19, 21-23, 25-27 are prepared analogously.

The compounds have the following structures

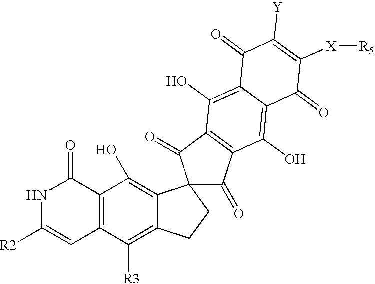

| Com- | | | | | UV/VIS |
|---|---|---|---|---|---|
| pound | R2 | R3 | X—R5 | Y | LC-MS |
| 1 | CHO | Br | N(CH₃)₂ | Br | M/e: 673 |
| | | | | | λₘₐₓ = 507.0 nm |
| 2 | (CH₃)₂CHON═CH— | Br | N(CH₃)₂ | Br | M/e: 730 |
| | | | | | λₘₐₓ = 504.0 nm |
| 3 | CH₃CH═CHCH═CH— | Br | NHCH₃ | Br | M/e: 696 |
| | | | | | λₘₐₓ = 506.0 nm |
| 4 | CH₃CH═CHCH═CH— | Br | N(CH₃)₂ | Br | M/e: 711 |
| | | | | | λₘₐₓ = 508.0 nm |
| 5 | CH₃CH═CHCH═CH— | Br | 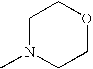 | Br | M/e: 753λₘₐₓ = 500.0 nm |
| 6 | CH₃CH═CHCH═CH— | Br | 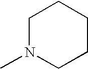 | Br | M/e: 751λₘₐₓ = 506.0 nm |
| 7 | CH₃CH═CHCH═CH— | Br | 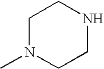 | Br | M/e: 752λₘₐₓ = 504.0 nm |
| 8 | CH₃CH═CHCH═CH— | Br | 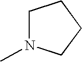 | Br | M/e: 737λₘₐₓ = 504.0 nm |
| 9 | CH₃ON═CH— | Br | N(CH₃)₂ | Br | M/e: 702 |
| | | | | | λₘₐₓ = 504.0 nm |
| 10 | CH₃CH═CHCH═CH— | Br | 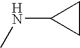 | Br | M/e: 723λₘₐₓ = 504.0 nm |
| 11 | CH₃ON═CH— | Br | NHCH₃ | Br | M/e: 688 |
| | | | | | λₘₐₓ = 504.0 nm |
| 12 | CH₃ON═CH— | Br | 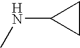 | Br | M/e: 714λₘₐₓ = 500.0 nm |
| 13 | (CH₃)₂CHON═CH— | Br | NHCH₃ | Br | M/e: 716 |
| | | | | | λₘₐₓ = 504.0 nm |
| 14 | (CH₃)₂CHON═CH— | Br | 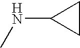 | Br | M/e: 728λₘₐₓ = 504.0 nm |
| 15 | CH₃CH═CHCH═CH— | Br | NHCH₂CH₂F | Br | M/e: 729 |
| | | | | | λₘₐₓ = 504.0 nm |
| 16 | CH₃CH═CHCH═CH— | H | OH | 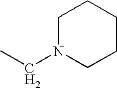 | M/e: 623λₘₐₓ = 500.0 nm |
| 17 | CH₃CH═CHCH═CH— | H | OH | —CH₂Net₂ | M/e: 611 |
| | | | | | λₘₐₓ = 500.0 nm |
| 18 | CH₃CH═CHCH═CH— | H | OH | CH₂NMe₂ | M/e: 583 |
| | | | | | λₘₐₓ = 504.0 nm |
| 19 | CH₃CH═CHCH═CH— | H | OH | 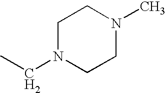 | M/e: 638λₘₐₓ = 500.0 nm |
| 20 | 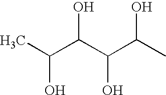 | H | OH | —CH₂NMe₂ | M/e: 651λₘₐₓ = 498.0 nm |
| 21 | CH₃CH═CHCH═CH— | I | N(CH₃)₂ | I | M/e: 805 |
| | | | | | λₘₐₓ = 506.0 nm |
| 22 | CH₃CH═CHCH═CH— | Br | NHCH₂CH═CH₂ | Br | M/e: 723 |
| | | | | | λₘₐₓ = 504.0 nm |
| 23 | CH₃CH═CHCH═CH— | Br | 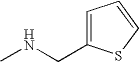 | Br | M/e: 779λₘₐₓ = 504.0 nm |
| 24 | CH₃CH═CHCH═CH— | I | NHCH₃ | I | M/e: 791 |
| | | | | | λₘₐₓ = 506.0 nm |
| 25 | CH₃CH═CHCH═CH— | I | 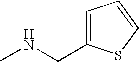 | I | M/e: 873λₘₐₓ = 504.0 nm |
| 26 | CH₃CH═CHCH═CH— | Br | OH | 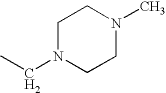 | M/e: 716λₘₐₓ = 502.0 nm |
| 27 | CH₃CH═CHCH═CH— | H | OH | CH₂NHCH₃ | M/e: 568 |
| | | | | | λₘₐₓ = 504.0 nm |

### Example 1a

### (8S)-4′,9,9′-trihydroxy-5-thiocyanato-6′-methoxy-1,1′,3,5′,8′-pentaoxo-1,1′,2,3′,5′,6,7,8′-octahydrospiro[cyclopenta[g]-isoquinoline-8,2′-cyclopenta[b]naphthalene]-3-carbaldehyde-O-methoxime (Compound 1)

19.0 mg (35.8 μmol) of fredericamycin aldehyde methoxime are dissolved under N₂ in 2 ml of acetic acid. After the addition of 15.2 mg (157.5 μmol) of potassium rhodanide, 3.6 μl (71.6 μmol) of bromine dissolved in 1 ml of acetic acid are added at 50° C. [122° F.]. The above-mentioned amount of potassium rhodanide/bromine at 50° C. [122° F.] is added each time at intervals of 1 hr, 2 hrs, 3.5 hrs and 5 hrs. After a total of 6 hrs, the reaction solution is dripped into 150 ml of water. This mixture is shaken out twice with chloroform, dried over sodium sulfate and concentrated until dry.

Yield: 7.0 mg (33% of the theoretical yield) of a red crystal powder. M/e=588, λₘₐₓ=502.0 nm.

### Example 2a

### (8S)-4′,9,9′-trihydroxy-5-thiocyanato-6′-methoxy-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 2)

20.0 mg (37.1 μmol) of fredericamycin are dissolved under N₂ in 2 ml of acetic acid. After the addition of 7.9 mg (81.4 μmol) of potassium rhodanide, 1.9 μl (37.1 μmol) of bromine dissolved in 0.5 ml of acetic acid are dripped in. After 3 hours, 39.5 mg (407.0 μmol) of potassium rhodanide and 9.5 μl (185.5 μmol) of bromine dissolved in 0.5 ml of acetic acid are added. This is heated to 50° C. [122° F.]. After 3 hours, the reaction mixture is added to 50 ml of water and the precipitate is aspirated. It is then washed with water and dried. The residue is picked up in chloroform and shaken out four times with water, then dried and concentrated.

Yield: 6.0 mg (27% of the theoretical yield) of a red crystal compound. M/e=597, λₘₐₓ=504.0 nm.

Compounds 3a and 4a are prepared analogously.

### Example 3a

### (8S)-4′,9,9′-trihydroxy-6′-azido-3-[(1E,3E)-penta-1,3-dienyl]-6,7-dihydrospiro[cyclopenta[g]-isoquinoline-8,2′-cyclopenta[b]-naphthalene]-1,1′-3′,5′,8′ (2H)-pentone (Compound 5)

10.0 mg (19.0 μmol) of hydroxy fredericamycin (demethylated fredericamycin) are dissolved under N₂ in 3 ml of dichloromethane. After the addition of 3.2 μl (19.0 μmol) of trifluormethane sulfonic acid anhydride and 2.3 μl (19.0 μmol) of 2,6-lutidine at 0° C. [32° F.], this mixture is stirred for another 10 minutes. It is then allowed to come to room temperature and 1.3 mg (19.0 μmol) of sodium azide are added. It is then stirred for 14 hours. Subsequently, the reaction solution is diluted with 20 ml of dichloromethane/1% trifluoroacetic acid. It is shaken out twice with water, the organic phase is dried over sodium sulfate and concentrated until dry. The remaining residue is purified by means of preparative HPLC (RP18, acetonitrile/water/trifluoroacetic acid).

Yield: 8.0 mg (76% of the theoretical yield) of a red solid. M/e=551.0; λₘₐₓ=504.0 nm.

### Example 4a

### (8S)-4′,9,9′-trihydroxy-5-dimethylaminomethyl-6′-methoxy-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 6)

10.0 mg (18.5 μmol) of fredericamycin are dissolved under N₂ in 2 ml of absolute dimethylformamide. After the addition of 36.6 mg (391.0 μmol) of N,N-dimethylmethylene ammoniumchloride in 1 ml of absolute dimethylformamide, the mixture is heated to 50° C. [122° F.]. After 24 hours, the reaction solution is placed into 70 ml of water/trifluoroacetic acid. The aqueous phase is extracted twice with dichloromethane. It is dried over sodium sulfate and concentrated. The remaining residue is purified by means of preparative HPLC (RP18, acetonitrile/water/trifluoroacetic acid).

Yield: 5.3 mg (48% of the theoretical yield) of a red solid. M/e=597.0; λₘₐₓ=504.0 nm.

### Example 5a

### (8S)-4′,9,9′-trihydroxy-5-dimethylaminomethyl-6′-methylamine-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 7)

10.0 mg (16.8 μmol) of 5-dimethylaminomethyl fredericamycin (Compound 25) are dissolved under N₂ in 1.2 ml of absolute dimethylformamide. After the addition of 200.0 μl (400.0 μmol) of methylamine (2M in methanol) and after 4 hours at 40° C. [104° F.], the reaction solution is placed into 60 ml of water/trifluoroacetic acid. The precipitate is aspirated, washed with water and dried. The residue is purified by means of preparative HPLC (RP18, acetonitrile/water/trifluoroacetic acid).

Yield: 4.2 mg (42% of the theoretical yield) of a red solid. M/e=596.0; λₘₐₓ=504.0 nm.

### Example 6a

### (8S)-4′,9,9′-trihydroxy-5-dimethylaminomethyl-6′-morpholino-3-[(1E,3E)-penta-1,3-dienyl]-6,7-dihydrospiro[cyclopenta[g]-isoquinoline-8,2′-cyclopenta[b]-naphthalene]-1,1′-3′,5′,8′(2H)-pentone (Compound 8)

5.0 mg (8.4 μmol) of 5-dimethylaminomethyl fredericamycin (Compound 25) are dissolved under N₂ in 0.5 ml of absolute morpholine and stirred for 1 hour at room temperature. The reaction solution is then added to 50 ml of water/trifluoroacetic acid. The precipitate is aspirated, washed with water and dried.

Yield: 1.8 mg (33% of the theoretical yield) of a red solid. M/e=652.0; λₘₐₓ=504.0 nm.

Compounds 3, 4 were prepared analogously,

The compounds have the following structures

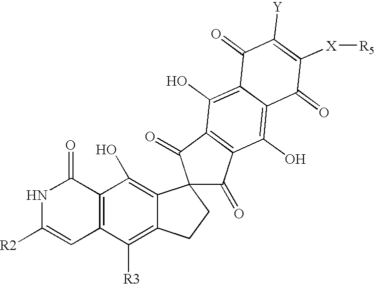

| Com- | | | | | UV/VIS |
|---|---|---|---|---|---|
| pound | R2 | R3 | X—R5 | Y | LC-MS |
| 1a | CH₃ON═CH— | SCN | OCH₃ | H | M/e = 588 λₘₐₓ = 502.0 nm |
| 2a | CH₃CH═CHCH═CH— | SCN | OCH₃ | H | M/e = 597 λₘₐₓ = 504.0 nm |
| 3a | (CH₃)₂CHON═CH— | SCN | OCH₃ | H | M/e = 616 λₘₐₓ = 500.0 nm |
| 4a | PhCH₂ON═CH— | SCN | OCH₃ | H | M/e = 664 λₘₐₓ = 504.0 nm |
| 20a | CH₃CH═CHCH═CH— | H | N₃ | H | M/e = 551 λₘₐₓ = 504.0 nm |
| 24a | CH₃CH═CHCH═CH— | CH₂NMe₂ | OCH₃ | H | M/e = 597 λₘₐₓ = 504.0 nm |
| 27a | CH₃CH═CHCH═CH— | CH₂NMe₂ | NHCH₃ | H | M/e = 596 λₘₐₓ = 504.0 nm |
| 28a | CH₃CH═CHCH═CH— | CH₂NMe₂ | 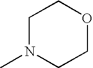 | H | M/e = 652 λₘₐₓ = 504.0 nm |

### Example A

### Water-Solubility of Fredericamycin Derivatives

The water-solubility of the various fredericamycin derivatives can be determined in a 0.9%-solution of NaCl having a pH value of 7.

### Example B

### Determination of the Efficacy of the Compounds on the Survival of Tumor Cells

### Cytotoxic Effect

The effect of the compounds on the survival of the human breast cancer cell line MCF7 was measured.

The cell line was analyzed at 37° C. [98.6° F.], 95% humidity and 5% CO₂ in RPMI Medium (Cambrex).

The cells are inoculated in a 96-well microtiter plate (Costar) at an initial density of 2400 cells per well and cultivated for 24 hours.

The compounds are dissolved in DMSO, diluted with cell medium and added to the wells.

The cells are incubated for another 48 hours at a concentration of the compounds between 2.4 nM and 10,000 nM at a volume of 50 μl.

50 μl of cell-titer Glo (Promega) are added to each well and the microtiter plate is incubated for 2 minutes at room temperature on a shaker and then left standing in the dark for 10 minutes.

The luminescence is measured with a microplate reader and is proportional to the number of surviving cells. The percentage of inhibition of the cell survival is calculated in comparison to (i) without cells and with compound (100% inhibition) and (ii) with cells and without compound (no inhibition).

The concentration of the half-maximum inhibition (IC₅₀) is determined with GraphPad Prism (GraphPad Software), whereby the controls are 0% and 100%.

The structures and the efficacy of the compounds according to the invention can be gleaned from the following table:

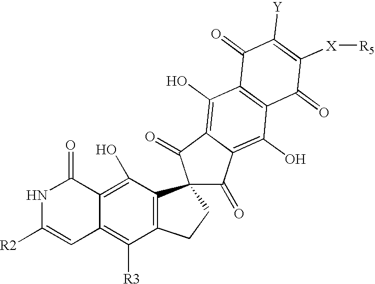

| Com- | | | | | mcf7 |
|---|---|---|---|---|---|
| pound | | | | | IC₅₀ |
| # | R2 | R3 | X-R5 | Y | (μM) |
| 7 | CH₃CH═CHCH═CH— | Br | NHCH₃ | Br | 0.19 |
| 13 | CH₃ON═CH— | Br | N(CH₃)₂ | Br | 0.30 |
| 8 | CH₃CH═CHCH═CH— | Br | N(CH₃)₂ | Br | 0.32 |
| 12 | CH₃CH═CHCH═CH— | Br | —N-pyrroridino | Br | 0.38 |
| 10 | CH₃CH═CHCH═CH— | Br | —N-piperidino | Br | 0.45 |
| 9 | CH₃CH═CHCH═CH— | Br | N-morpholino | Br | 0.67 |
| 30 | CH₃CH═CHCH═CH— | Br | NHCH₂CH═CH₂ | Br | 0.60 |
| 31 | CH₃CH═CHCH═CH— | Br | NHCH₂-2-thiophene | Br | 0.70 |

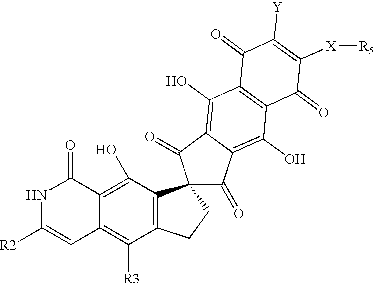

| | | | | | mcf7 |
|---|---|---|---|---|---|
| Com- | | | | | IC₅₀ |
| pound | R2 | R3 | X—R5 | Y | (μM) |
| 7a | CH₃CH═CHCH═CH— | CH₂N(CH₃)₂ | NHCH₃ | H | 0.66 |

1. A compound having the general Formula Ia or Ib: 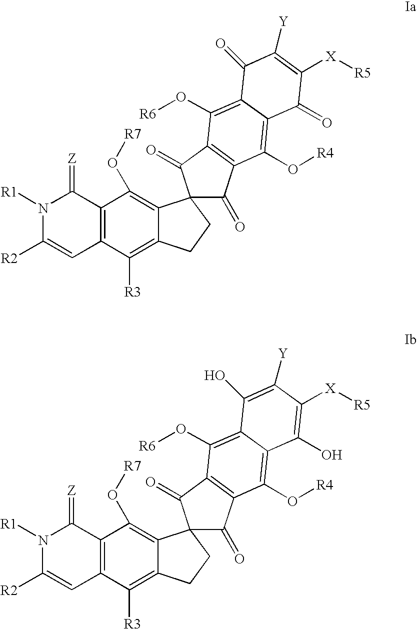
wherein
R1 is H, C₁-C₆-alkyl, cycloalkyl, or C₁-C₄-alkyl-cycloalkyl,
R2 is H, C₁-C₁₄-alkyl, C₂-C₁₄-alkenyl, aryl, C₁-C₄-alkyl-aryl, heteroaryl, C₁-C₄-alkyl-heteroaryl, C₂-C₄-alkenyl-heteroaryl, cycloalkyl, C₁-C₄-alkyl-cycloalkyl, heterocycloalkyl, C₁-C₄-alkyl-heterocycloalkyl, CₘH₂ₘ₊ₒ₋ₚYₚ, (CH₂)ᵣCH₂NHCOR21, (CH₂)ᵣCH₂OCOR21 (CH₂)ᵣCH₂NHCSR21, (CH₂)ᵣCH₂S(O)nR21 (CH₂)ᵣCH₂SCOR21, (CH₂)ᵣCH₂OSO₂—R21, (CH₂)ᵣCHO, CH₂—O—N═CH-aryl, CH₂—O—N═CH-hetaryl, CH₂—O—N═CH—R21, CH₂—O—N═CR21R22, CH₂—O—N═CH-cycloalkyl, CH═N—S-aryl, CH═N—S-hetaryl, (CH₂)ᵣCH═NOH, (CH₂)ᵣCH(OH)R21, —(CH₂)ᵣCH═NOR21, (CH₂)ᵣCH═NOCOR1, (CH₂)ᵣCH═NOCH₂CONR21R22, (CH₂)ᵣCH═NOCH(CH₃)CONR21R22, —(CH₂)ᵣCH═NOC(CH₃)₂CONR21R22, (CH₂)ᵣCH═N—NHCO—R23, (CH₂)ᵣCH═N—NHC(O)NH—R23, (CH₂)ᵣCH═N—NHC(S)NH—R23, (CH₂)ᵣCH═N—NHC(NH)NH—R23, (CH₂)ᵣCH═N—NHC(NH)—R23, (CH₂)ᵣCH═N—NHCO—CH₂NHCOR21, (CH₂)ᵣCH═N—O—CH₂NHCOR21, (CH₂)ᵣCH═N—NHCS—R23, (CH₂)ᵣCH═CR24R25 (trans or cis), (CH₂)ᵣCOOH, (CH₂)ᵣCOOR21, (CH₂)ᵣCONR21R22, —(CH₂)ᵣCH═NR21, (CH₂)ᵣCH═N—NR21R22, 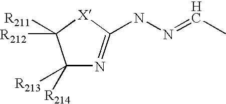
(CH₂)ᵣCH═N—N—(C₁-C₃-alkyl-NX′R211R212R213R214), —(CH₂)ᵣCH═N—NHSO₂-aryl, or —(CH₂)ᵣCH═N—NHSO₂-heteroaryl,
wherein m=1 to 6, for o=1, p=1 to 2m+o; for m=2 to 6, o=−1, p=1 to 2m+o;
for m=4 to 6, o=−2, p=1 to 2m+o;
Y, independent of each other, is selected from the group consisting of halogen, OH, OR21, NH₂, NHR21, N21R22, SH, and SR21;
n=0, 1, or 2;
X′═NR215, O, S,
R211, R212, R213, R214, and R215, independent of each other, are H or C₁-C₆-alkyl): and
r=0, 1, 2, 3, 4, or 5;
R21, R22 independent of each other, are C₁-C₁₄-alkyl, C₁-C₁₄-alkanoyl, C₁-C₆-alkylhydroxy, C₁-C₆-alkylamino, C₁-C₆-alkylamino-C₁-C₆-alkyl, C₁-C₆-alkylamino-di-C₁-C₆-alkyl, cycloalkyl, C₁-C₄-alkyl-cycloalkyl, heterocycloalkyl, C₁-C₄-alkylheterocycloalkyl, aryl, aryloyl, C₁-C₄-alkyl-aryl, heteroaryl, heteroaryloyl, C₁-C₄-alkyl-heteroaryl, cycloalkanoyl, C₁-C₄-alkanoyl-cycloalkyl, heterocycloalkanoyl, C₁-C₄-alkanoyl-heterocycloalkyl, C₁-C₄-alkanoyl-aryl, C₁-C₄-alkanoyl-heteroaryl, or a mono- and di-sugar radical that is linked via a C-atom that would carry an OH group in the sugar,
the sugars, independent of each other, are selected from the group consisting of glucuronic acid and its stereoisomers on all optical C-atoms, aldopentoses, and aldohexoses, including their deoxy compounds;
R23 independent of R21, has the same meanings as R21 or CH₂-pyridinium salts, CH₂-tri-C₁-C₆-alkyl ammonium salts,
R24 independent of R21, has the same meanings as R21 or H, CN, COCH₃, COOH, COOR21, CONR21R22, NH₂, NHCOR21;
R25 independent of R21, has the same meanings as R21 or H, CN, COCH₃, COOH, COOR21, CONR21R22, NH₂, NHCOR21;
R24, R25 together are C₄-C₈-cycloalkyl,
R3 is H, F, Cl, Br, I, OH, OR31, NO₂, NH₂, NHR31, NR31R32, NHCHO, NHCOR31, NHCOCF₃, CH₃₋ₘHalₘ, OCOR31, SCN, CN, N₃, CH₂NR331R332, CH₂OH, CH₂OR33, CH₂SR33, C₂-C₁₄-alkyl, C₂-C₁₄-alkenyl, C₂-C₁₄-alkinyl, C₂-C₁₄-alkyl, C₂-C₁₄-alkenyl, C₂-C₁₄-alkinyl, aryl, C₁-C₄-alkyl-aryl, heteroaryl, C₁-C₄-alkyl-heteroaryl, whereby the aryls or heteroaryls can be substituted with another aryl, C₁-C₄-alkyl-aryl, O-aryl, C₁-C₄-alkyl-O-aryl, heteroaryl, C₁-C₄-alkyl-heteroaryl, O-heteroaryl or C₁-C₄-alkyl-O-heteroaryl; cycloalkyl, C₁-C₄-alkyl-cycloalkyl, heterocycloalkyl, C₁-C₄-alkyl-heterocycloalkyl, CₘH₂ₘ₊ₒ₋ₚYₚ, CH₂NHCOR31, CH₂NHCSR31, CH₂S(O)nR31 with n=0, 1, 2, CH₂SCOR31, CH₂OSO₂—R31, CHO, CH═NOH, CH(OH)R31, —CH═NOR31, —CH═NOCOR31, —CH═NOCH₂CONR31R32, —CH═NOCH(CH₃)CONR31R32, —CH═NOC(CH₃)₂CONR31R32, —CH═N—NHCOR33, —CH═N—NHCO—CH₂NHCOR31, —CR═N—O—CH₂NHCOR31, —CH═N—NHCS—R33, —CH═CR34R35 (trans or cis), COOH, COOR31, CONR31R32, —CH═NR31, —CH═N—NR31R32, 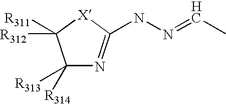
—CH═N—NHSO₂-aryl, —CH═N—NHSO₂-heteroaryl, SCN, CN, N₃, CH₂NR331R332, or CH₂SR33,
Hal=Cl or F;
m=1, 2, 3)
R331, R332, independent of each other, have the same meaning as R33:
with m=2 to 6, for o=1, −1, p=1 to 2m+o: for m=4 to 6, o=−3, p=1 to 2m+o;
Y, independent of each other, is selected from the group consisting of halogen, OH OR31, NH₂, NHR31, NR31R32, SH, and SR31;
X′═NR315, O, or S,
R311, R312, R313, R314, R315, independent of each other, are H or C₁-C₆-alkyl;
R331, R332, independent of each other, have the same meaning as R33;
R31, R32 independent of each other, are C₁-C₁₄-alkyl, C₁-C₁₄-alkanoyl, C₁-C₆-alkylhydroxy, C₁-C₆-alkylamino, C₁-C₆-alkylamino-C₁-C₆-alkyl, C₁-C₆-alkylamino-di-C₁-C₆-alkyl, cycloalkyl, C₁-C₄-alkyl-cycloalkyl, heterocycloalkyl, C₁-C₄-alkylheterocycloalkyl, aryl, aryloyl, C₁-C₄-alkyl-aryl, heteroaryl, heteroaryloyl, C₁-C₄-alkyl-heteroaryl, cycloalkanoyl, C₁-C₄-alkanoyl-cycloalkyl, heterocycloalkanoyl, C₁-C₄-alkanoyl-heterocycloalkyl, C₁-C₄-alkanoyl-aryl, C₁-C₄-alkanoyl-heteroaryl, or a mono- and di-sugar radical that is linked via a C-atom that would carry an OH group in the sugar,
the sugars, independent of each other, are selected from the group consisting of glucuronic acid and its stereoisomers on all optical C-atoms, aldopentoses, and aldohexoses, including their deoxy compounds;
R33 independent of R31, has the same meanings as R31 or CH₂-pyridinium salts, CH₂-tri-C₁-C₆-alkylammonium salts,
R34 independent of R31, has the same meanings as R31 or H, CN, COCH₃, COOH, COOR21, CONR31R32, NH₂, NHCOR31;
R35 independent of R31, has the same meanings as R31 or H, CN, COCH₃, COOH, COOR31, CONR31R32, NH₂, NHCOR31;
R34, R35 together are C₄-C₈-cycloalkyl,
R5 sad is H, C₁-C₆-alkyl, cycloalkyl, C₁-C₄-alkyl-cycloalkyl, heterocycloalkyl, C₁-C₄-alkyl-heterocycloalkyl, aryl, C₁-C₄-alkyl-aryl, heteroaryl, or C₁-C₄-alkylheteroaryl,
R4, R6, R7 independent of each other, are H, C₁-C₆-alkyl, or CO—R41;
R41 independent of R21, has the same meanings as R21;
X is O, S, NH, N—R8, wherein R8, independent of R5, has the same meaning as R5, or R5 and R8, together with N, form a 4-, 5-, 6-, 7- or 8-membered heterocycloalkyl ring that can optionally contain another heteroatom selected from the group consisting of N, O, and S,
or X—R5 together are H, F, Cl, Br, I, N3;
Y is F, Cl, Br, I, N₃, CN, CH₂NRY1RY2, CH₂OH, CH₂ORY₁, CH₂SRY1, SCN, aryl, hetaryl NRY1RY2, together with N, form a 4-, 5-, 6-, 7- or 8-membered heterocycloalkyl ring that can optionally contain another heteroatom selected from the group consisting of N, O, and S, and when X—R5 together are F, Cl, Br, I, or N₃, Y can also be H, W—R51, wherein W═O, S, NH, or N—R81, and R81 and R51, independent of each other, have the same meaning as R5, or R51 and R81, together with N, form a 4-, 5-, 6-, 7- or 8-membered heterocycloalkyl ring that can optionally contain another heteroatom selected from the group consisting of N, O and S, or H, W—R51, wherein W═O, S NH or N—R81, wherein R81 and R51, independent of each other, have the same meaning as R5, or R51 and R81, together with N, form a 4-, 5-, 6-, 7- or 8-membered heterocycloalkyl ring that can optionally contain another heteroatom selected from the group consisting of N, O, and S,
RY1, RY2, independent of each other, have the same meaning as R23;
Z is O, S, or NR9, wherein R9 is H or C₁-C₆-alkyl,
their stereoisomers, tautomers and their physiologically compatible salts or inclusion compounds.

2. The compounds according to claim 1, whereby Formula Ia or Ib have the stereochemistry of Formula IIa or IIb

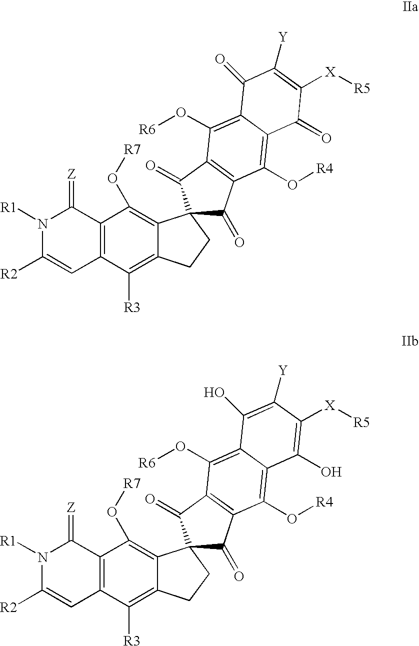

3. The compound having the general having Formulas Ia, Ib, IIa or IIb according to claim 1, in which the radicals R, aside from R3, have the meanings given above and R3, in comparison to when R3 equals H, increases the water-solubility—with the retention of all of the other radicals—by a factor of at least two.

4. The compound having the general Formulas Ia, Ib, IIa or IIb according to claim 1, in which the radicals R, aside from R2, have the meanings given in the preceding claims and R2, in comparison to when R2 equals CH═CH—CH═CH—CH₃, increases the water-solubility—with the retention of all of the other radicals—by a factor of at least two.

5. The compound according to claim 1, wherein
R1 is H, C₁-C₅-alkyl, or cycloalkyl,
R2 is H, C₁-C₁₄-alkyl, C₂-C₁₄-alkenyl, aryl, C₁-C₄-alkyl-aryl, heteroaryl, C₁-C₄-alkyl-heteroaryl, C₂-C₄-alkenyl-heteroaryl, cycloalkyl, C₁-C₄-alkyl-cycloalkyl, heterocycloalkyl, C₁-C₄-alkyl-heterocycloalkyl, CₘH₂ₘ₊ₒ₋ₚYₚ, (CH₂)ᵣCH₂NHCOR21, (CH₂)ᵣCH₂OCOR21, (CH₂)ᵣCH₂NHCSR21, (CH₂)ᵣCH₂S(O)nR21, (CH₂)ᵣCH₂SCOR21, (CH₂)ᵣCH₂OS₂—R21, (CH₂)ᵣCHO, CH₂—O—N═CH-aryl, CH₂—O—N═CH-hetaryl, CH₂—O—N═CH—R21, CH₂—O—N═CR21R22, CH₂—O—N═CH-cycloalkyl, CH═N—S-aryl, CH═N—S-hetaryl, (CH₂)ᵣCH═NOH, (CH₂)ᵣCH(OH)R21, —(CH₂)ᵣCH═NOR21, (CH₂)ᵣCH═NOCOR21, (CH₂)ᵣCH═NOCH₂CONR21R22, (CH₂)ᵣCH═NOCH(CH₃)CONR21R22, —(CH₂)ᵣCH═NOC(CH₃)₂CONR21R22, (CH₂)ᵣCH═N—NHCO—R23, (CH₂)ᵣCH═N—NHC(O)NH-1R23, (CH₂)ᵣCH═N—NHC(S)NH—R23, (CH₂)ᵣCH═N—NHC(NH)NH—R23, (CH₂)ᵣCH═N—NHC(NH)—R23, (CH₂)ᵣCH═N—NHCO—CH₂NHCOR21, (CH₂)ᵣCH═N—O—CH₂NHCOR21, (CH₂)ᵣCH═N—NHCS—R23, (CH₂)ᵣCH═CR24R25 (trans or cis), (CH₂)ᵣCOOH, (CH₂)ᵣCOOR21, (CH₂)ᵣCONR21R22, —(CH₂)ᵣCH═NR21, (CH₂)ᵣCH═N—NR21R22, 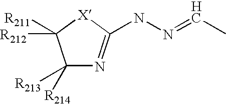
(CH₂)ᵣCH═N—N—(C₁-C₃-alkyl-NX′R211R212R213R214), —(CH₂)ᵣCH═N—NHSO₂-aryl, or —(CH₂)ᵣCH═N—NHSO₂-heteroaryl,
with m=1 to 6, for o=1, p=1 to 2m+o; for m=2 to 6, o=−1, p=1 to 2m+o; for m=4 to 6, o=−2, p=1 to 2m+o;
Y, independent of each other, is selected from the group consisting of halogen, OH, OR21, NH₂, NHR21, NR21R22, SH, and SR21);
n=0, 1, or 2;
X′═NR215, O, or S;
R211, R212, R213, R214, R215, independent of each other, are H or C₁-C₆-alkyl);
r=0, 1, 2, 3, 4, or 5;
R21, R22 independent of each other, are C₁-C₆-alkyl, cycloalkyl, aryl, C₁-C₄-alkyl-aryl, heteroaryl, or C₁-C₄-alkyl-heteroaryl;
R23 independent of R21, has the same meanings as R21 or CH₂-pyridinium salts, CH₂-tri-C₁-C₆-alkyl ammonium salts,
R24 independent of R21, has the same meanings as R21 or H, CN, COCH₃, COOH, COOR21, CONR21R22, NH₂, NHCOR21;
R25 independent of R21, has the same meanings as R21 or H, CN, COCH₃, COOH, COOR21, CONR21R22, NH₂, NHCOR21;
R24, R25 together are C₄-C₈-cycloalkyl,
R3 is H, F, Cl, Br, I, OH, OR31, NO₂, NH₂, NHR31, NR31R32, NHCHO, NHCOR31, NHCOCF₃, CH₃₋ₘHalₘ, OCOR31, SCN, CN, N₃, CH₂NR331R332, CH₂OH, CH₂OR33, CH₂SR33, C₂-C₁₄-alkyl, C₂-C₁₄-alkenyl, C₂-C₁₄-alkinyl, C₂-C₁₄-alkyl, C₂-C₁₄-alkenyl, C₂-C₁₄-alkinyl, aryl, C₁-C₄-alkyl-aryl, heteroaryl, C₁-C₄-allyl-heteroaryl, whereby the aryls or heteroaryls can be substituted with another aryl, C₁-C₄-alkyl-aryl, O-aryl, C₁-C₄-alkyl-O-aryl, heteroaryl, C₁-C₄-alkyl-heteroaryl, O-heteroaryl or C₁-C₄-alkyl-O-heteroaryl; cycloalkyl, C₁-C₄-alkyl-cycloalkyl, heterocycloalkyl, C₁-C₄-alkyl-heterocycloalkyl, CₘH₂ₘ₊ₒ₋ₚYₚ, CH₂NHCOR31, CH₂NHCSR31, CH₂S(O)nR31 with n=0, 1, 2, CH₂SCOR31, CH₂OSO₂—R31, CHO, CH—NOH, CH(OH)R31, —CH═NOR31, —CH═NOCOR31, —CH═NOCH₂CONR31R32, —CH═NOCH(CH₃)CONR31R32, —CH═NOC(CH₃)₂CONR31R32, —CH═N—NHCOR33, —CH═N—NHCO—CH₂NHCOR31, —CH—N—O—CH₂NHCOR31, —CH═N—NHCS—R33, —CH—CR34R35 (trans or cis), COOH, COOR31, CONR31R32, —CH═NR31, —CH—N—NR31R32, 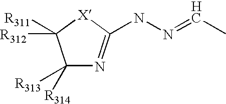
—CH═N—NHSO₂-aryl, or —CH═N—NHSO₂-heteroaryl,
Hal=Cl or F,
m=1, 2, 3;
R331, R332, independent of each other, have the same meaning as, R33);
with m=2 to 6, for o=1, −1, p=1 to 2m+o; for m=4 to 6, o=−3, p=1 to 2m+o;
Y, independent of each other, is selected from the group consisting of halogen, OH, OR31, NH₂, NHR31, NR31R32, SH, and SR31;
X′═NR315, O, or S;
R311, R312, R313, R314, R315, independent of each other, are H or C₁-C₆-alkyl);
R331, R332 independent of each other, are C₁-C₄-alkyl,
R31, R32 independent of each other, are C₁-C₄-alkyl,
R5 is H, C₁-C₃-alkyl, cycloalkyl, or heterocycloalkyl,
R4, R6, R7 independent of each other, are H, C₁-C₅-alkyl, or CO—R41,
R41 independent of R21, has the same meanings as R21;
X is O, S, NH, N—R8, wherein R8, independent of R5, has the same meaning as R5 or R5 and R8, together with N, form a 6-membered heterocycloalkyl ring that can optionally contain another heteroatom selected from the group consisting of N and O,
or X—R5 together are H,
Y is H, F, Cl, Br, I, or N₃,
Z is O, S, or NH.

6. The compound according to claim 1 in the form of inclusion compounds with cyclodextrin.

7. A pharmaceutical drug containing compounds according to claim 1 in addition to the usual carriers and auxiliaries.

8-12. (canceled)

13. A method of preparing a pharmaceutical composition comprising mixing a compound of claim 1 with galenic auxiliaries or carriers.

14. A method of treating tumors that can be treated through the inhibition of topoisomerase I or II, comprising administering to a patient in need of such treatment an effective amount of a compound of claim 1.

15. The method of claim 14, wherein said tumor is leukemia, lung cancer, melamona, prostate cancer or colon tumors.

16. A method of treating parasites comprising administering to a patient in need of such treatment an effective amount of a compound of claim 1.

17. A method of treating immunosuppression comprising administering to a patient in need of such treatment an effective amount of a compound of claim 1.

18. A method of treating neurodermatitis comprising administering to a patient in need of such treatment an effective amount of a compound of claim 1.
